(19) United States
(12) Patent Application Publication
(10) Pub. No.: US 2024/0122628 A1
(43) Pub. Date: Apr. 18, 2024

(54) OSTEOSYNTHESIS DEVICE, IN PARTICULAR FOR SPINAL TREATMENT, AND ATTACHMENT SYSTEM FOR THIS

(71) Applicant: ORTHO HUB VENTURES UG (haftungsbeschränkt), Filderstadt (DE)

(72) Inventor: Frank HEUER, Filderstadt (DE)

(21) Appl. No.: 18/555,217

(22) PCT Filed: Apr. 13, 2022
(86) PCT No.: PCT/EP2022/059942

§ 371 Date: Oct. 12, 2023

(30) Foreign Application Priority Data
Apr. 13, 2021 (DE) 10 2021 001 896.6
Apr. 12, 2022 (DE) 10 2022 108 980.0

(51) Int. Cl. A61B 17/70

(52) U.S. Cl. CPC A61B 17/7037; A61B 17/7032; A61B 2017/00477

(57) ABSTRACT

An attachment system for an osteosynthesis device for spinal treatment is disclosed having a main body including a first receiving region which is designed for receiving a connecting element, a second receiving region which is designed for receiving a bone anchor, a third receiving region having a linear transverse opening, the transverse opening having a first opening at its first end and a second opening at its second end, an axial opening for receiving and guiding a positioning element, and the device further having a fixing element and a positioning element.

### FIELD OF THE INVENTION

The invention relates to an osteosynthesis device, in particular for spinal treatment, and to an attachment system for the same.

### STATE OF THE ART

Various osteosynthesis devices for the care of the spine, such as for example pedicle screws, are known in the prior art. Such osteosynthesis devices are used to correct spinal misalignments or stabilize fractures by inserting and attaching the osteosynthesis devices into the vertebral bone and then connecting them together via longitudinal rods, or so-called connecting rods, in order to fix the vertebrae together in a desired position. In this case, the longitudinal rods are mounted on the osteosynthesis devices with the aid of locking elements, such as for example grub screws or other closure elements, and fixed in a non-slip manner. Pedicle screws are preferably used as osteosynthesis devices, which have a bone anchor that is pivotably mounted in at least one plane with a fork head and is angularly stable when the grub screw is fixed. Bone screws with a ball head are preferably used as bone anchors. Osteosynthesis devices with bone anchors and fork heads are usually mounted in such a way that the bone anchor in a proximal direction in the fork head is guided through the distal opening of the fork head. This only works if the outer diameter of the bone anchor shaft is smaller than the ball head diameter of the bone anchor and the outer diameter of the bone anchor shaft is smaller than the diameter of the distal opening of the fork head. Assembly is problematic if the outer diameter of the bone anchor shaft is larger than the opening diameter of the fork head and/or of the ball head diameter of the bone anchor.

A pedicle screw (DE102011053295A1) is known from the prior art, which can be temporarily locked and thus allows an extended range of applications in the care of spinal instabilities. This makes it possible to insert polyaxially movable pedicle screws into the vertebral bone in any orientation and then the user can temporarily clamp the angle between the fork head and the bone anchor. With a temporarily blocked polyaxiality, it is possible that corrective forces are induced on the bone anchor via the fork head and this has a direct effect on the setting and position of the vertebral bones. Without this temporary clamping, such corrective manoeuvres cannot be carried out or can only be carried out with difficulty. With such an arrangement, it is necessary for the thrust piece to be permanently clamped with the aid of an instrument and for the instrument to be attached to the screw at all times in order to maintain the compression force necessary for the temporary clamping. In some spinal correction manoeuvres, such as for example the correction of deformities and scoliosis, there is no room for such instruments. Therefore, it would be desirable to provide a screw implant that is independently able to maintain the temporary clamping once the instruments are removed.

From Application DE102018102173B3 a pedicle screw anchor is known in which the temporary compression force is permanently maintained by providing a detachable pin element. However, this construction requires a lever-like actuation of the thrust piece to generate the temporary clamping. This results in an increased combined compression and bending stress of the thrust piece, which leads to a mechanical limitation of the maximum temporary clamping effect. Furthermore, it is necessary for the thrust piece to be dimensioned in its material thickness in such a way that these loads do not have a destructive effect. As a result, such a pedicle screw is larger in its overall height than a regular pedicle screw. It is therefore desirable that the temporary clamping effect is not induced via the thrust piece itself, but acts directly on the bone anchor head region. Thus, the thrust piece is mechanically decoupled during the temporary clamping and the osteosynthesis device can provide reserves with regard to the maximum clamping effect.

Furthermore, it can be seen that none of these temporarily clampable pedicle screw designs is able to accommodate screw shafts coming from the distal direction. They can only be used with bone anchors in which the outer diameter of the bone anchor shaft is smaller than the diameter of the distal opening of the fork head.

Osteosynthesis devices or pedicle screws known from the prior art are accordingly inflexible and insecure with regard to positioning and fixing and require a lot of installation space during implantation and adjustment, but also with regard to permanent retention in the patient. In addition, they are difficult to handle, especially for the surgeon.

### DESCRIPTION OF THE INVENTION

The object of the present invention is therefore to make it possible for osteosynthesis devices to be simple, flexible and safe to handle, in particular simple, flexible and safe to position and fix, and to have a small installation space.

The object is achieved by an attachment system according to claim 1, an osteosynthesis device with an attachment system according to claim 1 and a bone anchor for an attachment system according to claim 1.

The object is further achieved by an osteosynthesis device according to claim 15 and by an osteosynthesis device according to claim 16.

Further features of the invention are contained in the dependent claims.

This makes it possible to provide a temporarily clampable osteosynthesis device, in particular a pedicle screw, which allows a bone anchor to be mounted coming from the distal direction and that the thrust piece is not loaded when the temporary clamping is actuated, but is only subjected to a compressive force when the osteosynthesis device is finally locked with the connecting rod and the closure element. This results in a modularity of the screw system, which has advantages with regard to the reduced capital commitment of the user, and an overall larger screw portfolio can be offered. Furthermore, the force-flow distribution in the case of the temporary clamping without the mechanical involvement of the thrust piece results in a significant gain in stability compared to previous designs, and the pedicle screw can be made smaller in its overall height. At the same time, the design according to the invention is intended to maintain the temporarily generated clamping independently, even if all instruments have been removed. In the following, components, attachment systems and osteosynthesis devices are depicted, by means of which a bone anchor can be fixed in its alignment or position. According to the invention, where this seems helpful for understanding, a distinction is made between pre-fixing and final fixing. According to the invention, pre-fixing or pre-fixed is understood to mean fixing the bone anchor in its alignment or position, with which it is achieved that during the surgical implantation of an attachment system or an osteosynthesis device, a temporary securing of the position or alignment of the bone anchor can be guaranteed, for example in order to connect the osteosynthesis device to further components, such as further osteosynthesis devices, or to insert and/or position them precisely. Conversely, according to the invention, the final fixing or finally fixed is understood to mean the fixing of the bone anchor, with which it is achieved that this, including, if appropriate, the attachment system or osteosynthesis device fixed to or with it, in particular also with respect to movement-induced loads, remains positionally stable in the patient.

Furthermore, below the spatial specification is used proximally, which means that the final alignment of the attachment system or of the osteosynthesis device is close to the surgeon. For components located conversely, the spatial specification is used distally, which in the final alignment of the attachment system or of the osteosynthesis device means a distance from the surgeon or, viewed from the proximal end, the opposite end of the attachment system or of the osteosynthesis device.

An attachment system according to the invention for an osteosynthesis device, in particular for spinal treatment, has a main body with a first receiving region, which is designed for receiving a connecting element, a second receiving region, which is designed for receiving a bone anchor, a third receiving region having a linear transverse opening, wherein the transverse opening has a first opening at its first end and a second opening at its second end, and an axial opening for receiving and guiding a positioning element. Furthermore, the attachment system according to the invention has a fixing element having a first fixing pin, and a load receiving region, wherein the first fixing pin is insertable into the transverse opening via the first opening and the fixing element is longer than the transverse opening, and wherein the first fixing pin has a contact surface at its end remote from the load receiving region, and a positioning element having a load introduction region, wherein the positioning element can be positioned and guided by means of the axial opening in such a way that the fixing element can be guided along the transverse opening in the direction of the second receiving region by means of the pressure exerted by the load introduction region on the load receiving region.

The transverse opening of the thrust piece can be designed, for example, as a cutout.

The load introduction region is also referred to hereinbelow as the contact region. The load receiving region is also referred to hereinbelow as the contact region. The contact surface is also referred to hereinbelow as the contact region.

In this case, if the bone anchor has an end designed as a ball head, the second receiving region can be designed as a ball head receiving region. The second receiving region is thereby preferably configured in such a way that the proximal end of the bone anchor can be received therein. This proximal end of the bone anchor is also referred to hereinbelow as the head region of the bone anchor.

The connecting element serves to connect several osteosynthesis devices and is in particular a connecting rod, particularly preferably a round rod.

The bone anchor can be configured, for example, as a bone screw or as a bone nail.

Preferably, the second receiving region is further designed for loosely fixing the bone anchor along its central axis. The central axis corresponds to the longitudinal axis of the bone anchor. As a result, unintentional detachment of the bone anchor from the attachment system, for example, the bone anchor falling out of the attachment system, can be prevented, thereby improving handling.

Preferably, the transverse opening has a constant cross section along its guide line. In this way, secure guidance of the fixing element in the transverse opening is made possible.

If there is a preferred alignment of the fixing element, it is the cross section of the part of the fixing element that is guided in the transverse opening and the cross section of the transverse opening are preferably respectively designed such that the fixing element can only be inserted into the transverse opening by means of the two cross sections in such a way that the fixing element is in the preferred alignment. This enables time-saving and safe handling.

Preferably, it is the cross section of the part of the fixing element that is guided in the transverse opening and the cross section of the transverse opening are designed to be complementary to one another. In this way, secure guidance of the fixing element with little play is made possible.

Preferably, the first opening is positioned on the inside. This means that the main body is configured in such a way that the fixing element can be inserted into the transverse opening via the second receiving region. Accordingly, the first opening is positioned and configured such that the second receiving region lies on the one side and the third receiving region lies on the other side. In this way, it can be achieved that in the configuration of the axial opening, it does not have to be provided that the fixing element has to be insertable into the transverse opening from the outside. In this way, a reduced installation space can be provided, whereby the attachment system on the patient can be used with little surgical effort.

Alternatively, the first opening can also be provided on the outside. This makes it possible to insert the fixing element into the transverse opening even if a bone anchor has already been received in the second receiving region.

The transverse opening is preferably configured and positioned in such a way that, depending on the geometry of the proximal end of the bone anchor, the fixing element can be guided in a load-optimized manner onto the proximal end of the fixing element, for example by avoiding the fixing element striking on an edge of the distal end of the bone anchor.

The transverse opening is preferably configured and positioned in such a way that, depending on the geometry of the proximal end of the bone anchor, the fixing element can be guided onto the proximal end of the bone anchor in such a way that the bone anchor is not deflected out of this desired alignment regardless of its alignment in the second receiving region. This can be achieved, for example, by guiding in a centred manner the fixing element on the distal end of the bone anchor in relation to the distal end of the bone anchor. This increases the flexibility of the attachment system and improves its handling.

As the fixing element is longer than the transverse opening, it is achieved that the first fixing pin can at least partially emerge from the transverse opening and be pressed onto the bone anchor received in the second receiving region. In this way, the bone anchor can be fixed in the second receiving region.

The fact that the fixing element is longer than the transverse opening can be achieved by configuring the fixing pin longer than the transverse opening. However, the transverse opening can also be configured in such a way that the load receiving region is at least partially inserted into it, so that the fixing pin does not necessarily have to be configured longer than the transverse opening. In this way, a particularly space-saving configuration can be achieved.

Preferably, the third receiving region is configured in such a way that the transverse opening is provided with an offset with respect to the central axis of the bone anchor received in the second receiving region in the neutral position, the offset being at least 20°, preferably 30° and particularly preferably 40° and a maximum of 80°, preferably 70° and particularly preferably 60°. The offset refers here to the fact that the fixing element is not pressed onto the bone anchor from proximal to distal, i.e. along the central axis of the bone anchor received in the second receiving region in the neutral position, but is offset from this orientation or axis. Preferably, the offset is then effected by rotating the longitudinal axis of the transverse opening about a point of rotation that is positioned substantially centrally in the distal end of the bone screw. This ensures that the bone anchor or its distal end is pressed against the edge of the second receiving region. In this way, an enhanced fixing effect is achieved. The neutral position of the bone anchor corresponds here to the position of the bone anchor and the main body with respect to one another, in which the central axis of the bone anchor runs essentially along the direction of the main body, starting from the proximal end of the first receiving region towards the second receiving region, i.e. directed distally.

Preferably, the distal end of the bone anchor is spherical, for example as a ball head, and the second receiving region is also spherical complementary thereto. This makes it possible to ensure that, even at small offset angles, the distal end of the bone anchor is pressed at least partially perpendicularly onto at least one surface portion of the second receiving region.

Preferably, the load introduction region and the load receiving region are designed to be complementary to one another. In this way, it can be achieved that the load to be transmitted by the positioning element to the fixing element is transmitted over as large a contact surface as possible, as a result of which the pressure exerted here on the contact surface in the process is minimized. This enables secure and robust guidance of the fixing element.

If the positioning element has a thread, that is, it is designed, by way of example, as a load screw, and the load introduction region is provided at the distal end of the positioning element, the load receiving region can be provided in a concave manner. In this way, the load can be reliably transmitted when the positioning element rotates.

Preferably, the contact surfaces of the load introduction region and of the load receiving region are oriented substantially perpendicular to the longitudinal axis of the transverse opening. As a result, it can be achieved that the load is exerted on the fixing element along the transverse opening, as a result of which the risk of tilting or wedging of the fixing element is minimized. This allows safe handling of the attachment system or of the osteosynthesis device.

Preferably, the attachment system further comprises a thrust piece, wherein the thrust piece has a first receiving region equivalent to the first receiving region of the attachment system, a second receiving region equivalent to the second receiving region of the attachment system and having a plurality of spring elements, and a third receiving region equivalent to the third receiving region of the main body and having a linear transverse opening, and wherein the thrust piece can be inserted into the main body via the first receiving region of the main body. Equivalently in this case it means that, in the inserted state of the thrust piece, the connecting element can be received in the attachment system via both first receiving regions, that is via the first receiving region of the main body and the first receiving region of the thrust piece, the bone anchor can be inserted via both the two second receiving regions, that is the second receiving region of the main body and the second receiving region of the thrust piece, along the central axis of which it can be loosely fixed, and the fixing element can be inserted via the first openings of both third receiving regions, that is via the first opening of the main body and via the first opening of the thrust piece, into the transverse openings thereof. The thrust piece then serves to enable a secure final fixation of the bone anchor and then a stabilization of the damaged region of the body is ensured. This final fixing is achieved by pressing the connecting element onto or into the first receiving region of the thrust piece in such a way that the second receiving region of the thrust piece is deformed so that the spring elements are moved toward one another. If a bone anchor or the proximal end of a bone anchor is now located in the second receiving region of the thrust piece, the spring elements are pressed onto this, resulting in a frictional connection or force closure between the second receiving region of the thrust piece and the bone anchor or its proximal end. The first receiving region of the thrust piece is also referred to hereinbelow as a stave bearing.

The second receiving region of the thrust piece is also referred to hereinbelow as the bone anchor head region. The spring element is also referred to hereinbelow as an arm or resilient arm.

It is also possible for such spring elements to be provided in the second receiving region of the main body, by means of which such a frictional connection or force closure is then achieved.

Preferably, the third receiving region of the thrust piece is configured in such a way that no force closure connection can result between the fixing element and the thrust piece, particularly preferably even in the state of the fixing element pressed onto the bone anchor. In this way, a secure pre-fixing of the bone anchor is made possible, while at the same time avoiding jamming or tilting of the thrust piece, so that ultimately a secure final fixation of the bone anchor is also made possible. In order to achieve this, it is possible, for example, to provide large-area recesses in the transverse opening of the thrust piece, which are preferably configured to be complementary to the first fixing pin or, if appropriate, complementary to further fixing pins.

Preferably, the thrust piece is further configured such that a first insertion position is made possible, in which the bone anchor can be inserted into the second receiving region of the main body, and a second insertion position is made possible, in which the bone anchor can no longer be removed from the main body. In this case, it is particularly preferably adjustable from proximal to distal that the thrust piece is inserted into the first receiving region of the main body, wherein the thrust piece is not displaced as far as the distal end of the first receiving region of the main body. Proceeding from this, the second insertion position can then be reached by inserting or pushing the thrust piece further distally into the first receiving region of the main body. During the displacement of the thrust piece from the first insertion position to the second insertion position, the spring elements of the thrust piece can be briefly radially deflected in order to subsequently pivot back into their neutral position. If the thrust piece has been displaced into the second insertion position, the distal ends of the spring elements block the distal end of the second receiving region of the main body, so that the bone anchor can no longer be removed distally from the main body. In this way, a loss prevention for the bone anchor is made possible, whereby a safe handling of the attachment system or of the osteosynthesis device is made possible. For this purpose, the second receiving region may be designed to taper in the distal direction.

Preferably, the axial opening has an inner thread and the positioning element has an outer thread, and the inner thread of the axial opening is designed to be complementary to the outer thread of the positioning element. This makes it possible to apply a load on the fixing element by screwing the positioning element. This enables a precise and secure fixation of the bone anchor. The outer thread of the positioning element is also referred to hereinbelow as the thread.

Preferably, the first receiving region of the main body further has an inner thread proximally. This makes it possible for the connecting element to be pressed into the first receiving region or into the first receiving regions by means of a screw, for example by means of a grub screw.

Preferably, the thread diameter of the axial opening and the thread diameter of the positioning element are smaller than the thread diameter of the first receiving region of the main body. In this way, a small space requirement of the attachment system can be achieved, whereby the tissue damage occurring in the patient during surgical implantation is minimized.

Preferably, the inner thread of the axial opening and the outer thread of the positioning element have a thread pitch that is smaller than the thread pitch of the inner thread of the first receiving region of the main body. In this way, the tightening torque to be applied to load the positioning element can be reduced, as a result of which the torsional load on the positioning element is reduced and, at the same time, a secure fixing of the bone anchor through the fixing element is made possible. This is particularly advantageous if the positioning element is designed with smaller dimensions, for example with a smaller circumference, than the grub screw to be received in the first receiving region of the main body, since the positioning element then has a lower torsional strength.

Preferably, the inner thread of the axial opening and the outer thread of the positioning element have a pitch of maximum 1.00 mm per revolution, preferably of less than 0.85 mm per revolution, particularly preferably of less than 0.70 mm per revolution, and of at least 0.1 mm per revolution, preferably 0.2 mm per revolution, particularly preferably 0.3 mm per revolution. In this way, it can be achieved that, on the one hand, the torsional load on the positioning element is reduced and, at the same time, the positioning element generates a significantly higher axial prestress in the axial opening, as a result of which the handling of the attachment system is improved.

Preferably, the inner thread of the axial opening extends substantially parallel to the central axis of the bone anchor received in the second receiving region in the neutral position. In this way, a space-saving design of the attachment element is made possible, whereby the tissue damage occurring during the surgical implantation of the osteosynthesis device is reduced. Furthermore, it is thereby made possible to actuate the positioning element and the grub screw received in the first receiving region of the main body in a similar manner and lying close to one another, whereby the handling is improved.

It is also possible for the axial opening to be aligned parallel to the main axis of the transverse opening of the main body. In this way, the force closure effect between the positioning element and the fixing element and ultimately between the fixing element and the bone anchor can be maximized, which in turn increases the reliability and stability of the attachment system.

It is further possible for the transverse opening of the main body to be aligned substantially perpendicular to the central axis of the bone anchor received in the second receiving region of the main body in the neutral position. In this way, a maximum clamping effect with respect to the bone anchor can be achieved.

The axial opening preferably has at least one compression opening, wherein the compression opening is positioned at the distal end of the inner thread, and wherein the compression opening is designed in such a way that it enables the outer thread of the positioning element to be compressed. In this way, in the sense of loss prevention, it is possible to prevent the positioning element from being automatically detached from the axial opening, for example, as a result of which the handling of the attachment system is improved. Compression is understood here to mean a local deformation of the outer thread of the positioning element, which is so intense that when the deformed outer thread section engages in an inner thread that is basically complementary to this outer thread, the deformed or compressed outer thread section is wedged in the inner thread.

It is also possible to compress the outer thread over the outer wall of the axial opening. However, there would be the risk that the axial opening would also be deformed, as a result of which the positioning element could possibly be completely clamped, i.e. neither rotated out of the axial opening nor rotated into it. It is therefore advantageous to configure the main body in such a way that a compression of the outer thread of the positioning element is locally limited without damaging the inner thread of the axial opening and, in addition, with respect to the positioning element, and this cannot be carried out in a manner that is substantially damaging.

The axial opening preferably has a plurality of compression openings, by way of example two, which are particularly preferably positioned opposite one another. In this way, a uniform compression is made possible without possibly damaging the surrounding structures.

Preferably, the inner thread formed in the axial opening ends distally in front of the compression opening or of the compression openings. In this way, even after the outer thread of the positioning element has been compressed, it is possible to screw it further into the axial opening in the distal direction, while at the same time preventing unscrewing in the proximal direction.

Preferably, the fixing element further comprises a second fixing pin. This makes it possible for the bone anchor to be clamped over two load points via the fixing element. As a result, the load points arise via the contact surfaces between the distal ends of the first and second fixing pin and the bone anchor. Starting from the respective contact surface, from the region located on the opposite side of the bone anchor and of the load introduction direction, a load direction or load axis arises in each case. If the fixed bone anchor is now loaded, for example during surgical implantation, there are sometimes different clamping resistances. If this load occurs along the load axis, a large clamping resistance arises. However, if the load occurs away from the load axis, for example in the form of a torsional load around the load axis, a low clamping resistance arises. This can lead to the clamping effect not being able to be provided to the required extent with regard to every possible load. It can also lead to the positioning element being actuated to an increased extent in order to be able to provide the required clamping effect with regard to every possible load, as a result of which the fixing element is then pressed to an increased extent onto the bone anchor. However, this increases the risk of material failure. If several load points are now made possible, this risk can in turn be minimized and at the same time a secure clamping effect can be achieved with regard to every possible load situation.

It is also possible to configure a single fixing pin in such a way that it has a contact surface which, when engaged with the bone anchor, results in a plurality of load axes. This can be achieved, for example, by providing two elevations spaced apart from one another on the contact surface.

The fixing element is preferably configured in such a way that, when the bone anchor is clamped through this, it achieves a three-point mounting or three-point load. In this way, with regard to any load situation of the bone anchor the secure clamping thereof is made possible in a simple manner.

The contact surface of the first fixing pin or optionally of the first and/or second fixing pin is preferably complementary to a corresponding contact surface of the bone anchor. In this way, a safe and material-friendly load transfer from the fixing pins to the bone anchor is made possible.

Particularly preferably, the contact surface of the first fixing pin or optionally of the first and/or second fixing pin is configured in such a way that the contact region between the first fixing pin or optionally the first and/or second fixing pin and the bone anchor is maximized. In this way, the corresponding friction surface is also maximized, which in turn maximizes the closure force effect.

Preferably, the attachment system is provided for a bone anchor with a ball head. In this way, a spherically concavely curved contact surface of the fixing pin adapted to this ball head can make it possible for the bone anchor to be securely pre-fixed regardless of its rotational position. The rotational position is understood to mean any possible position of the bone anchor resulting geometrically from the main body and the proximal end of the bone anchor.

Preferably, the approximate diameter of the spherically concavely curved contact surface of the first fixing pin or optionally of the first and/or second fixing pin is smaller than the diameter of the proximal end of the bone anchor designed as a ball head. This makes it possible for the resulting contact surface to be additionally subjected to shear stress, for example in the form of a crimping, when the first fixing pin or optionally the first and/or second fixing pin is pressed onto the proximal end of the bone anchor. Furthermore, despite the bending of the distal end of the first fixing pin or optionally of the distal ends of the first and/or second fixing pin increasing with increasing pressing pressure, it is possible for load to be transmitted over as large a region as possible. Furthermore, this makes it possible for the main load-bearing regions of the resulting contact surfaces to be displaced further radially outward with respect to the fixing pin, as a result of which an increased clamping resistance is generated, in particular with respect to a rotational load about the corresponding load axis. The approximate diameter is here understood to be the diameter of the sphere or of the circle that would result if the spherical-concave curvature were extended until hitting the starting point again.

Preferably, the approximate diameter of the spherically concavely curved contact surface of the first fixing pin or optionally of the first and/or second fixing pin is maximum 1.00 mm, preferably 0.50 mm, particularly preferably 0.20 mm and at least 0.01 mm, preferably 0.02 mm, particularly preferably 0.05 mm larger than the diameter of the proximal end of the bone anchor designed as a ball head.

Preferably, a spring element is arranged in the linear transverse opening of the thrust piece, and this spring element is placed in particular centrally in the linear transverse opening. In this way, it is made possible for the first and second fixing pin to be guided respectively past the spring element into engagement with the bone anchor without the spring element arranged in the linear transverse opening of the thrust piece coming here into force closure connection with the first and/or second fixing pin. In this way, it is possible to ensure a secure pre-fixing of the bone anchor by means of the fixing element without making therewith a final fixing of the bone anchor via the connecting element more difficult or preventing it.

Preferably, the spring element arranged in the linear transverse opening is designed to widen in the distal region. In this way, it can be achieved that when the thrust piece is inserted and the fixing element is inserted into its transverse opening, the thrust piece is prevented from unintentionally exiting the main body in the proximal direction. In this case, the fixing element additionally acts as a loss prevention device for the thrust piece. Furthermore, this maximizes the potential contact region between the spring elements and the proximal end of the bone anchor, thereby maximizing the clamping effect of the final fixation.

Preferably, the contact surface of the first fixing pin or optionally of the first and/or second fixing pin has a locally increased surface roughness. In this way, the frictional effect and thus the pre-fixing effect can be increased. The surface roughness can be produced, for example, by roughening or by providing notches and/or teeth.

Preferably, the proximal end of the bone anchor has a locally increased surface roughness. In this way, the frictional effect and thus the effect of the pre-fixing as well as the final fixing can be increased.

Preferably, the contact surface of the thrust piece has a locally increased surface roughness. In this way, the frictional effect and thus the effect of the final fixation can be increased.

The first receiving region is preferably designed as a fork head, in particular as a U-shaped fork head, which has a first fork leg and a second fork leg. In this way, a weight-optimized and space-optimized design is made possible, whereby, by way of example, the handling of the attachment system or of the osteosynthesis device is improved, in particular during surgical implantation in a patient.

Preferably, the first receiving region of the main body has a thread. This makes it possible to press the connecting element onto the bone anchor by means of a screw, for example by means of a space-saving grub screw, in particular indirectly via the thrust piece onto the bone anchor.

If the first receiving region is designed as a fork head, both fork legs can have a corresponding threaded section. The first fork leg then preferably has a threaded section complementary to the threaded section of the second fork leg, wherein both threaded sections together form the thread of the first receiving region of the main body or of the fork head. Furthermore, the fork legs and thus also the threaded sections formed therein are preferably arranged opposite one another. In this way, on the one hand, a secure tightening of the connecting element is made possible and, on the other hand, a further reduction in weight and installation space is made possible.

Preferably, the third receiving region and the axial opening are arranged on the first fork leg. In this way, the required installation space can be reduced, which improves handling.

Preferably, the proximal end of the third receiving region of the main body follows the distal end of the axial opening from proximal to distal. In this way, the necessary installation space can be reduced. Furthermore, this makes it possible to insert the fixing element from radially outwardly to radially inwardly into the transverse opening of the main body and, if appropriate, into the transverse opening of the thrust piece, as a result of which handling is improved.

Preferably, the axial opening is designed and positioned and/or the positioning element is designed in such a way that the positioning element can be positioned so that its distal end can protrude distally out of the axial opening in such a way that the radially outer end of the transverse opening of the main body is covered radially outward. As a result, a loss prevention can be achieved radially outward by means of the positioning element for the fixing element.

An osteosynthesis device according to the invention, in particular for spinal treatment, has a bone anchor and an attachment system according to the invention.

An alternative osteosynthesis device according to the invention, in particular a polyaxial pedicle screw, consists of a bone anchor having a head, a fork head that is U-shaped in a side view, a thrust piece located therein, an inner-lying fixing element guided in a transverse opening, and a detachably connected positioning element for actuating the fixing element, wherein the fixing element is suitable for temporarily clamping the bone anchor head region in all degrees of freedom without thereby a thrust piece being necessary or, if present, not being loaded.

In the preferred embodiment, it is possible to induce the clamping effect indirectly via a positioning element or directly via the fixing element without the connecting rod or grub screw being present. Since this is not a final clamping with an inserted connecting rod, this type of clamping is called temporary clamping. With the temporary clamping, it is possible for the user to convert a polyaxial screw in a desired angular position into a monoaxial screw during surgery. This means that all rotational degrees of freedom of a polyaxial screw are temporarily blocked. The screw behaves monoaxially. This allows the user to manipulate the vertebra to be treated both translationally and rotationally until he inserts a connecting rod in the desired end position and fixes it with the grub screw. Such correction manoeuvres are not possible with a polyaxial screw, since a correction manoeuvre initiated on the outside of the patient side results in a free movement of the polyaxial ball joint and is therefore not transmitted to the vertebra. This only works with deactivated rotational degrees of freedom in the ball joint, i.e. it is temporarily clamped.

Regardless of the temporary clamping, after implantation of the osteosynthesis device in the bone, a connecting rod must be inserted and the osteosynthesis device finally fixed in all degrees of freedom with the aid of a closure element. This takes place by screwing the closure element tightly. When the closure element is tightly fastened, an axial compression force is transmitted from the closure element on the connecting rod, and the latter presses on the rod supporting points of the thrust piece and generates a minimal relative movement of the thrust piece further distally, so that the bone anchor is clamped angularly stable in the ball seat. Optimally, two or more osteosynthesis devices are connected to one another with the aid of a connecting rod.

The fork head is here configured in such a way that a connecting rod can be inserted and fixed to the fork head with a locking element. As already mentioned, this achieves an angularly stable clamping between the bone anchor head region and the fork head. The angle stable clamping with the aid of the locking element and the angularly stable clamping with the aid of the fixing element work independently of one another in the structure according to the invention. They can be activated separately but also in combination.

In a preferred embodiment of the osteosynthesis device, the fork head has a through opening and, in the proximal direction, forms two fork legs with an inner-lying thread for a locking element. In the distal direction, a ball head receiving region is provided in the through opening, in which a bone anchor is pivotably mounted.

Bone screws that can be screwed to a bone are preferably used as bone anchors. However, hooks, blade-like anchors, clamps, nails and differently designed bone anchors can also be used. The essential features of the bone anchor are a ball-like head, a neck region, and a region that can be anchored or attached to the bone. This patent application is intended to deal mainly with the fork head, and bone anchors are intended to be understood to mean all conceivable elements that can be connected to a bone.

An essential feature is that the centre of the inner-lying thread and the centre of the ball head receiving region define the position and orientation of the central axis. An axial opening for the positioning element is provided laterally and at a distance that is greater than the diameter of the locking element. The axial opening is arranged mainly parallel to the central axis. The positioning element can thus be driven from the same direction as the locking element with the aid of instruments. In the axial opening, the positioning element is guided in a length-adjustable manner. In addition to openings completely enclosed by material, the term axial opening also refers to partial openings or partial cutouts such as, for example, c-shaped cutouts transversely to the central axis (103).

An additional opening, a transverse opening, is provided transversely to the axial opening and the central axis. The transverse opening establishes a connection between the axial opening and the through opening of the fork head. In this transverse opening, the fixing element is movably guided along the transverse opening axis. With the induction of a compression force along the transverse opening axis, the fixing element clamps the head region of the bone anchor in an angularly stable manner in the fork head.

The temporary clamping or compression force is preferably generated by a positioning element, which is guided in the axial opening, by adjusting the positioning element. The compression force is transmitted or deflected here to the fixing element, resulting in a compression force along the transverse opening axis, which leads to the temporary clamping of the bone anchor head region in the fork head.

Alternatively, it is also conceivable that the temporary clamping or the induction of a compression force can take place via the fixing element itself. For this purpose, it would be necessary, for example, for the fixing element and the transverse opening to be in engagement with one another with the aid of a threaded section, thus enabling a longitudinal adjustment.

A characteristic feature of the osteosynthesis device is that the transverse opening axis of the fixing element is directed approximately toward the virtual centre of the ball head receiving region and intersects with the central axis. As a result, the compression force can be directed toward the pivoting centre of the bone anchor in the fork head and an optimal clamping can be achieved.

Optimally, the fork head provides a lateral bulge or material thickening in order to receive therein the axial opening, transverse opening and the elements necessary for actuation (positioning and fixing element).

In a preferred embodiment, the fork head has a thrust piece which has a distally directed contact region towards the bone anchor head region and a proximally directed stave bearing. In the region of the bone anchor head region, a lateral opening or a partial cutout is provided, in which the fixing element is arranged in a freely movable manner. This allows the fixing element to be activated without the thrust piece being loaded.

In a preferred embodiment, the fork head may be mounted with bone anchors coming from the distal direction. Due to this advantageous arrangement of the components, the bone anchors can be mounted relatively easily with the fork head by placing or pressing them thereon. The bone anchor can also be removed again by means of an aid, such as for example a release instrument. The osteosynthesis device according to the invention can thus be configured modularly by the user and assembled in the operating room at a later time than that of production. This makes it possible, for example, for the bone anchor to be anchored or screwed individually into the bone first, and then for the fork head to be attached to the already implanted bone anchor. This has the great advantage that after the implantation of the bone anchor, the surgeon has significantly more space and a better view in the surgical field compared to the otherwise fully implanted pedicle screws.

The advantage is that, on the one hand, larger bone anchors, i.e. bone anchors with a larger outer diameter, than the distal inner diameter of the fork head can be mounted. On the other hand, the bone anchor portfolio can be minimized because the user can combine the fork head and bone anchor during surgery instead of resorting to a prefabricated oversized portfolio. Such a portfolio must be in stock with the user and thus significantly more capital is tied up than with the modular version according to the invention would require.

For a modular design of the osteosynthesis device, it is advantageous for the thrust piece to have open slots in the distal direction, and for at least three resilient arms to be designed on the head receiving region as a result. The resilient arms can deflect radially outwards and thus enclose the bone anchor head region. As a result, a bone anchor coming from the distal direction can be clipped into the thrust piece. The thrust piece forms a cone at least in sections at the distal end of the outer side. At least one inner cone section is defined in the fork head at the level of the ball receiving region, which leads congruently to the cone of the thrust piece and, when the locking element is actuated, to the angularly stable clamping of the bone anchor head region with the fork head. Here, too, it is essential for the thrust piece to provide a passage opening for the fixing element, so that the thrust piece is not loaded when the temporary clamping is activated.

Provided at the proximal fork head portion is a circumferential groove having a hook-like profile that provides a rear grip for an instrument. Representatively, differently designed groove profiles or other retaining features, such as for example openings, are conceivable, which provide a rear grip for an instrument.

At the proximal end of the fork head, there may be further and detachable portions with a threaded region that allow repositioning the connecting rod. It is also conceivable that a sleeve-like access formed by two longer legs is provided, as is used for minimally invasive access. The detachable leg extensions may thereby optionally be connected to one another at the proximal end. By detachable connection are meant, for example, predetermined breaking points which are suitable for removing the extensions after the connecting rod has been finally fixed.

All metal alloys that are known and accepted as orthopaedic implant material are suitable for use as a material. These include, for example, titanium, cobalt-chromium and stainless steel alloys. If the conventional production of the fork head and of the locking ring is not possible or only possible with the highest technological effort, additive manufacturing is the means of choice. Additive manufacturing of metal alloys, also known as 3D printing, uses the laser or electron beam melting process.

### BRIEF DESCRIPTION OF THE FIGURES

FIG. 1a shows an oblique view of the osteosynthesis device according to the invention.

FIG. 1b shows an oblique view of the osteosynthesis device according to the invention with the positioning element detached.

FIG. 2 shows an exploded view of the osteosynthesis device according to the invention consisting of a fork head, a fixing element, bone anchor and thrust piece.

FIG. 3 shows a side view of the mounted osteosynthesis device according to the invention with a sectional view.

FIG. 4 shows an illustration of the components required to actuate the temporary clamping.

FIG. 5a, b show two different positions S1, S2 of the positioning element.

FIG. 6a shows an oblique view and FIG. 6b shows the associated exploded view of an alternative embodiment in which a mounting of the bone anchor from the distal direction is possible.

FIG. 7 presents a side view of the mounted osteosynthesis device according to the invention from FIGS. 6a and 6b with a corresponding sectional view.

FIG. 8 shows the fully implanted osteosynthesis device in an oblique view.

FIG. 9 shows an alternative embodiment of an implanted osteosynthesis device in which the positioning element remains in the patient.

FIG. 10a shows an attachment system in an isometric view.

FIG. 10b shows an osteosynthesis device with the attachment system depicted in FIG. 10a in an isometric view.

FIG. 11 shows the attachment system depicted in FIGS. 10a and 10b in an exploded view.

FIG. 12a shows the osteosynthesis device depicted in FIG. 10b in a side view with a sectional plane.

FIG. 12b shows the osteosynthesis device depicted in FIGS. 10b and 12a in a sectional view along the sectional plane also depicted in FIG. 12a.

FIG. 13a shows a fixing element with a first fixing pin and a second fixing pin in an isometric view.

FIG. 13b shows the fixing element depicted in FIG. 13a in a side view.

FIG. 13c shows the fixing element depicted in FIGS. 13a and 13b in a plan view.

FIG. 13d shows the fixing element depicted in FIGS. 13a to 13c from below.

FIG. 14a shows a positioning element in engagement with the fixing element depicted in FIGS. 13a to 13d and a bone anchor in an isometric view.

FIG. 14b shows a fixing element with a first fixing pin in engagement with a head region of a bone anchor in a plan view.

FIG. 14c shows the fixing element depicted in FIGS. 13a to 13d in a plan view in the engagement position depicted in FIG. 14a with the head region of the bone anchor.

FIG. 15 shows the attachment system depicted in FIG. 10a in another isometric view.

FIG. 16a shows the fixing element depicted in FIGS. 13a to 13d in engagement with a thrust piece in an isometric view.

FIG. 16b shows the fixing element depicted in FIG. 16a in the engagement position with the thrust piece, also depicted in FIG. 16a, in a side view with a sectional plane.

FIG. 16c shows the fixing element depicted in FIGS. 16a and 16b in the engagement position, likewise depicted in FIGS. 16a and 16b, with the thrust piece in a sectional view along the sectional plane likewise depicted in FIG. 16b.

FIG. 17 shows the osteosynthesis device depicted in FIGS. 10b, 12a, and 12b with a connecting element in an isometric view.

### DESCRIPTION OF THE PREFERRED EMBODIMENTS

If reference is made below by means of reference numerals to features specifically depicted in the figures, the inventive teaching described in this context is not to be understood as being limited to the specific embodiment depicted in each case. Rather, the following description is to be interpreted taking into account the above general principles of the inventive teaching. This applies in particular to the following sections 1 to 30 relating to an osteosynthesis device 1 for treating the spine.

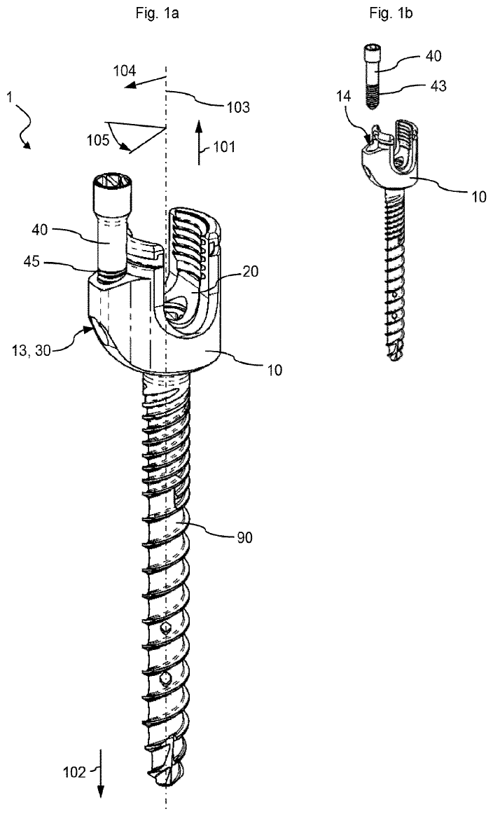

An osteosynthesis device 1 for treating the spine is described, wherein more than one osteosynthesis device 1 is used to connect one or more vertebrae to one another with the aid of connecting rods 50 and thus to stabilize the spine. For the osteosynthesis device 1, in particular for the fork head 10, coordinate references to be associated spatially are defined, such as the proximal direction 101, the distal direction 102, which extend along a central axis 103. Starting from the central axis 103 outwards, the radial extent 104 is defined and the circumferential extent 105 is defined by a constant radius and a variable circumferential angle (FIG. 1).

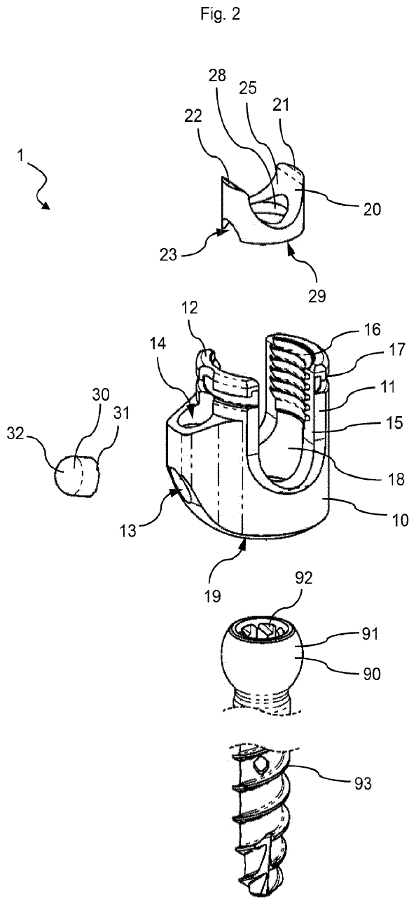

FIGS. 1a, 1b and FIG. 2 show the osteosynthesis device 1 according to the invention, for the treatment of the spine, consisting of a fork head 10 which is U-shaped in a side view and has a through opening 18, and the fork head 10 has two fork legs 11, 12 in the proximal direction 101 with an inner-lying thread 16, and a connecting rod 50 can be received therein, and a ball head receiving region 19 is provided in the fork head 10 in the distal direction 102 in the through opening 18, and a bone anchor 90 is pivotably mounted therein, wherein the centre of the inner-lying thread 16 and the centre of the ball head receiving region 19 define a central axis 103 and at least one leg 11, 12 provides an axial opening 14, arranged parallel to the central axis 103, for a positioning element 40. In the fork head 10, a transverse opening 13 is provided, which communicates with the axial opening 14 and with the through opening of the fork head 18. In this transverse opening 13, a fixing element 30 is movably guided along an axis 130. With the induction of a compression force along the transverse opening axis 130, the fixing element 30 clamps the head region 91 of the bone anchor 90 in an angularly stable manner in the fork head 10. When the compression force on the fixing element 30 is released, the head region of the bone anchor 91 in the fork head 10 becomes movable again.

The bone anchor 90 preferably has a head region 91 with a tool attachment point 92 located therein and has a bone thread 93 in the distal direction 102.

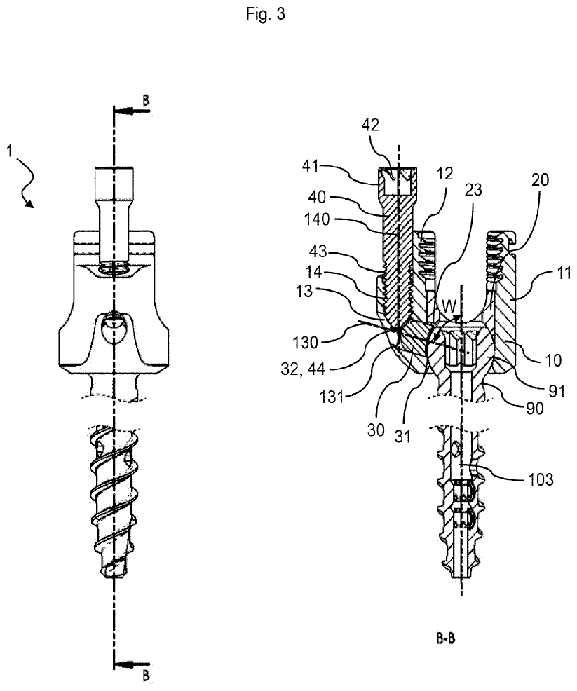

The osteosynthesis device 1 preferably has a pin-shaped positioning element 40, which has a tool attachment point 42 in the proximal direction 101 (FIG. 3). The positioning element 40 is designed to be detachable and, if necessary, can also be removed (FIG. 1b). With the positioning element 40 removed and the fixing element 30 not temporarily clamped, this osteosynthesis device 1 behaves like a polyaxial pedicle screw.

FIG. 1a and FIG. 2 also illustrate that the fork head 10 of the osteosynthesis device 1 has a thrust piece 20 and that the thrust piece has a through opening 28, a distally directed contact region towards the bone anchor head region 29, a proximally directed stave bearing 25, which is delimited by two legs 21, 22 and has a lateral opening or partial cutout 23 in the region of the bone anchor head region 29, in which the fixing element 30 is arranged so as to be freely movable. If a connecting rod 50 is inserted into the U-shaped fork opening 15, the connecting rod 50 is in direct contact with the stave bearing of the thrust piece 25. If the locking element 60 is now fixed with the fork head 10, a compression force is transmitted from the locking element 60 to the connecting rod 50, and from the latter to the thrust piece 20, 25, and from the thrust piece 20 to the bone anchor head region 29, 91, and the bone anchor 90 against the distal ball seat region 19. This induced compression force then leads to the clamping of the polyaxiality.

FIG. 2 shows the preferred structure of the fixing element 30. It is depicted here that the fixing element 20 is preferably designed as a round rod and is movably mounted in a concentric transverse opening 13. Alternative embodiments of the fixing element 20, which are not shown here, are also conceivable, such as, for example, the fixing element being configured as a triangle, quadrilateral or polygon in a sectional representation transverse to the transverse opening axis 130, or else as a triangle or polygon with transitional roundings, or as a solid round with lateral flattenings, which serve to prevent rotation of the fixing element 30. It is also conceivable for the fixing element 30 to change its geometry along the transverse opening axis 130 to provide engagement features for captive elements.

It can also be inferred from FIG. 2 that the fork head 10 has projections, openings, grooves, bars, profiles or other features 17 that are suitable for gripping, engaging or engaging from behind with an instrument. If the positioning element 40 is part of an instrument, this instrument binding feature 17 can be used to induce a tensile force on the fork head 10, which acts as an antagonist to the induction of a compression force via the positioning element 40.

FIG. 2 also shows, if an axial opening 14 is provided in a fork head leg 11, 12, that it is advantageous for the fork head 10 to have a lateral material bead, which merges into one of the legs 11 or 12, so that the axial opening 14 can be produced at all.

FIG. 3 shows the osteosynthesis device 1 according to the invention in a side view and in section. It can be seen that the axis of the fixing element 130 is directed approximately toward the virtual centre of the ball head receiving region 19 and intersects with the central axis 103. In addition, the transverse opening axis 130 also intersects the axial opening axis for the positioning element 140. Thus, the compression force is aligned with the centre of the ball-like bone anchor head region 91, so that a similarly large contact region 31, 91 is present even when the bone anchor 90 is pivoted. Furthermore, it is advantageous if the axis of the fixing element 130 is arranged at an angle W between 10° and 90°, preferably between 30° and 85°, preferably between 60° and 80°, with respect to the central axis 103. As a result, when the compression force is induced by the fixing element 30, the bone anchor head region 91 is not only pushed in the opposite direction or against the opposite inner wall of the fork head 18, but is also partially forced into the ball seat 19. Thus, the bone anchor 90 is centred in the fork head 10 in the ball seat 19 under the action of the force.

FIG. 2 also shows that the opening of the fixing element 13 along the fixing element axis 130 has, at least at one point, an indent, undercut, a pin, step, deformable region or taper 131 which is suitable for holding the fixing element 30 captively in this opening 13. For the assembly of the osteosynthesis device 1, the fixing element 13 is fitted into the transverse opening 13 coming from the central through opening 18. Subsequently, the head region 91 of the bone anchor 90 is placed into the through opening 18 in the ball seat 19 and the fixing element 30 is thus held captively in the transverse opening 13.

Alternatively, it is also conceivable that the transverse opening 13 for the fixing element 30 has an inner thread at least in sections, so that the fixing element 30 can itself generate the compression force by rotation. Alternatively, a sectional inner thread can also serve to secure the fixing element 30 against loss, in that the thread represents a barrier to be bridged.

The osteosynthesis device 1 is configured in such a way that a connecting rod 50 can be inserted and by means of a locking element 60 can be fixed to the fork head 10, thereby achieving an angularly stable clamping between the bone anchor head region 91 and the fork head 10, and the angularly stable clamping with the aid of the locking element 60 and the angularly stable clamping with the aid of the fixing element 30 can be activated independently of one another and can also be combined with one another.

In FIG. 3 the essential characteristic feature can also be seen, namely that when the bone anchor head region 91 is temporarily clamped solely by the fixing element 30, the thrust piece 20 is always unloaded. A force is applied to the thrust piece only by inserting and fixing a connecting rod 50, 60. As a result, the fork head and the thrust piece can absorb a higher mechanical load and the thrust piece can be provided with significantly less material than comparable designs, which in turn has a positive effect on the overall height of the osteosynthesis device 1. In osteosynthesis devices 1 for the spine, the smallest possible overall height is important in order to best adapt to the anatomical conditions of the patient.

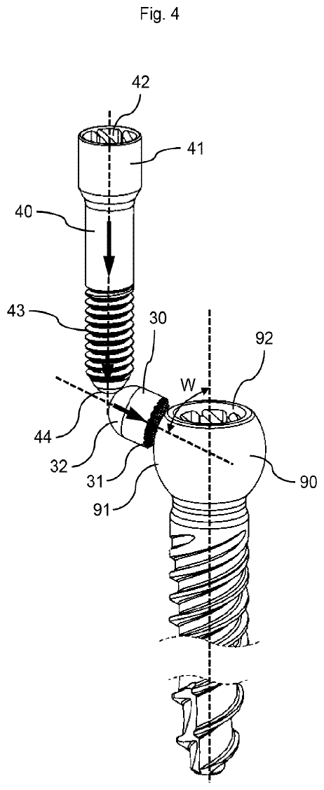

FIG. 4 shows a reduced structure in which only the elements generating the temporary clamping are shown. The remaining elements have been hidden.

By adjusting the positioning element 40, a compression force can be generated, which is transmitted or deflected to the fixing element 30, and thereby a compression force is generated along the transverse opening axis 130, which leads to the temporary clamping of the bone anchor head region 91 in the fork head 10.

In this case, the positioning element 40 is guided in the axial opening 14 of the fork head 10 that is hidden here. If the positioning element 40 is part of the implant, it is advantageous if the axial opening 14 for the positioning element 40 has an inner thread at least in sections. Being engaged with this, it is necessary for the positioning element 40 itself to have a thread 43 at least in sections. As a result, it is possible for the induced compression force to be maintained when the positioning element 40 is screwed in or fastened. If the positioning element 40 is part of an instrument, the positioning element 40 itself does not have to have a thread, since the compression force to be induced is generated by the instrument.

For the deflection of the compression force from the positioning element 40 to the fixing element 30, it is advantageous if the fixing element 30 has a radially outwardly directed contact region 32, which is in direct contact with a distal contact region 44 of the positioning element 40, and this contact 32, 44 is configured in such a way that a compression force along the positioning element axis 140 is diverted into a compression force along the fixing element axis 130. In this case, the radially outwardly directed contact region 32 of the fixing element 30 can be provided convexly in sections in at least one side view (FIG. 4). Alternatively, it is also conceivable for the radially outwardly directed contact region 32 of the fixing element 30 to have a bevel in at least one side view. In the preferred embodiment, it is provided that the distal contact region 44 of the positioning element 40 is convex, at least in sections.

The radially inwardly directed contact region 31 of the fixing element 30, which is responsible for the temporary clamping, abuts directly against the head region 91 of the bone anchor 90. It is advantageous if this contact region 31 is concavely shaped at least in sections in a side view or approximates at least a section of the outer surface of the bone anchor head region 91. For an optimized clamping effect, it is advantageous if the radially inwardly directed contact region 31 of the fixing element 30 has an increased roughness, notches or teeth (FIG. 4).

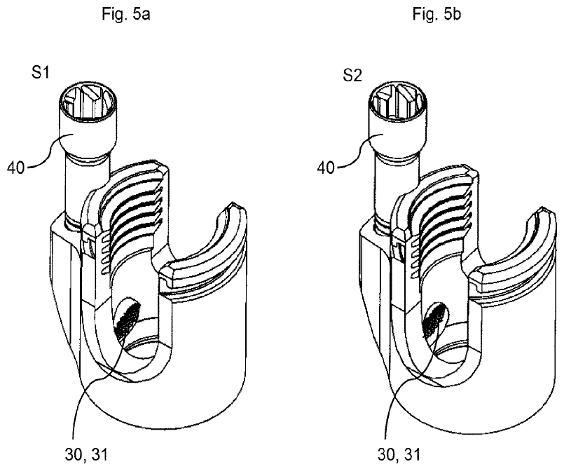

From FIG. 5 it can be inferred that the fixing element 30 can assume a position S1 along the transverse opening axis 130, in which a compression force is transmitted on the bone anchor 90, so that the bone anchor 90 is held angularly stable in the ball seat 19, and the fixing element 30 can assume a second position S2, in which the bone anchor 90 is movably held in the ball seat 19. These positions of the fixing element 30 can be adjusted by adjusting the positioning element 40. In this case, there is a position S1 in which the positioning element 40 moves the fixing element 30 radially inwards for temporary clamping, and a position S2 of the positioning element in which the fixing element is adjusted or moved radially outwards in such a way that free polyaxiality of the bone anchor arises.

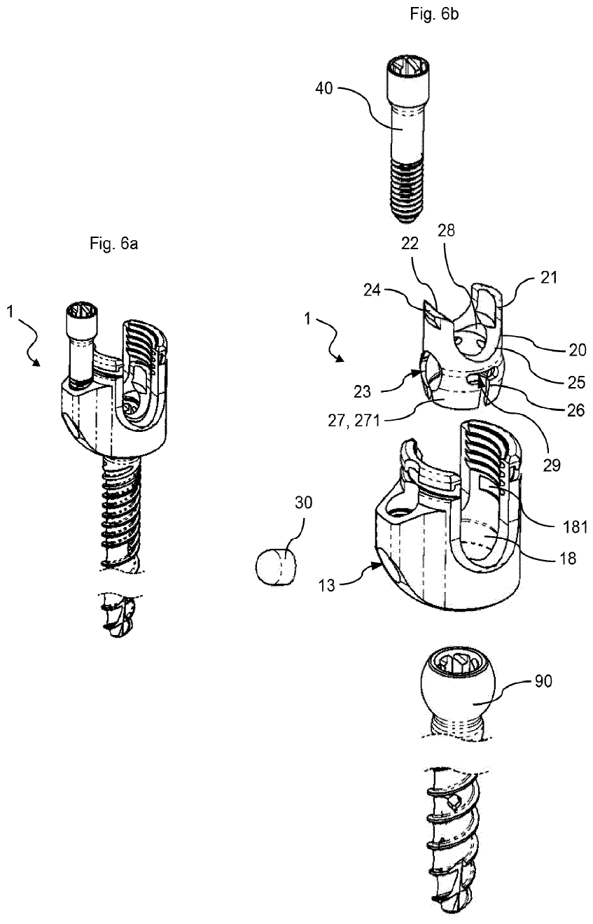

FIGS. 6a and 6b show a further preferred embodiment of the osteosynthesis device 1 according to the invention. Here, the thrust piece 20 has open slots 26 in the distal direction 102. As a result, at least three resilient arms 27 are formed on the head receiving region 29, wherein the resilient arms 27 describe a cone 271 on their outer side at least in sections, and the bone anchor 90 can be fitted into the fork head 10 coming from the distal direction 102. As a result, bone anchors 90 with a larger outer diameter can be mounted with the fork head 10.

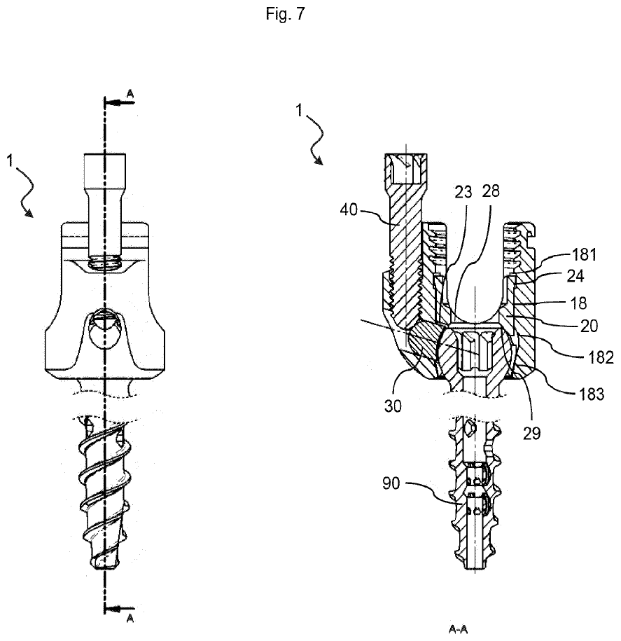

So that the thrust piece can be optimally clamped within the fork head 10, at least one inner cone section 183 is defined in the ball receiving region 19, which is provided congruently with the cone 271 of the thrust piece 20 and, when the locking element 60 is actuated, leads to the angularly stable clamping of the bone anchor head region 91 with the fork head 10 (FIG. 6a,b and FIG. 7). Here, too, it is necessary for the thrust piece to have a lateral opening or an at least partial cutout 23 through which the fixing element 30 is guided and can move therein. This ensures that the thrust piece 20 is not loaded when the temporary clamping is activated.

It can be inferred in FIG. 7 that the fork head legs 11, 12 each provide a supporting surface 181 with an undercut, which is effective in the proximal direction 101, and the thrust piece 20 has radially outwardly directed projections 24 at the proximal end, the projections 24 being designed to be resilient radially inwards, so that the thrust piece 20 coming from the proximal direction 101 can be fitted into the fork head 10. In this case, the projections of the thrust piece 24 latch with the supporting surfaces 181 and the thrust piece 20 is secured in the proximal direction 101 but not subjected to force. In FIG. 7, it can also be seen that the fork head 10 in the through opening 18 has a region which, at least in sections, has a larger inner diameter 182 than the core diameter of the thread 16.

As a result, if the thrust piece is not yet fully latched to the fork head 10 in the final position, it is possible for the resilient cone region 27 of the thrust piece in the fork head 10 to be provided with corresponding space for spreading apart at the level of the inner diameter widening 182 for assembly with the bone anchor head region 91.

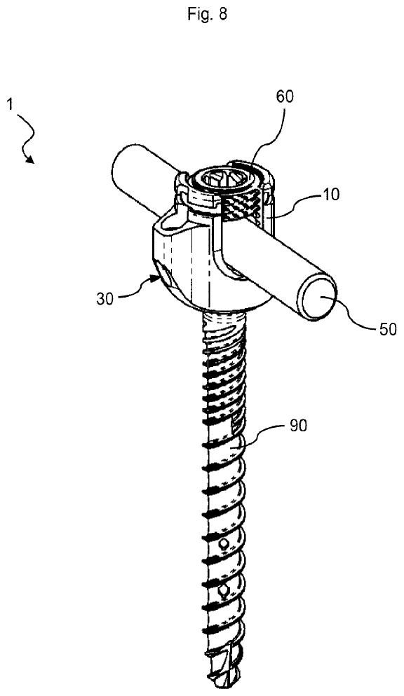

In FIG. 8 it is depicted that when the positioning element 40 is part of the osteosynthesis device 1 and has a predetermined breaking point 45, only a part of the positioning element 40 remains in the patient after the final locking with the connecting rod 50 and the locking element 60. The same picture is obtained if the positioning element 40 is part of an instrument and is removed from the patient after the final locking with the connecting rod 50 and the locking element 60.

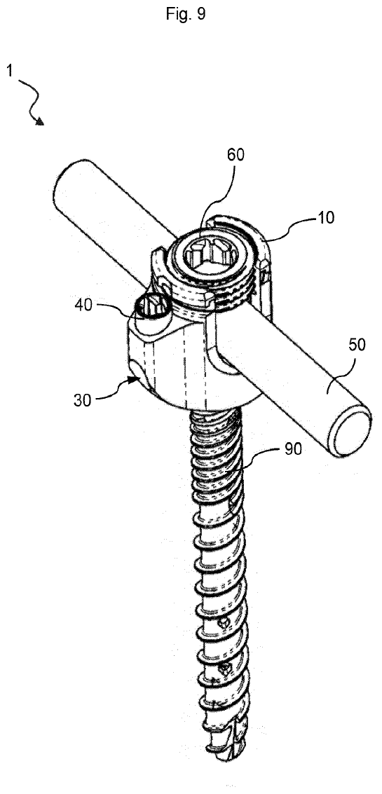

Alternatively, the positioning element 40 can also be provided completely as part of the osteosynthesis device 1. After the final locking with the connecting rod 50 and the locking element 60, it remains in the patient (FIG. 9). It is advantageous then if the positioning element 40 does not protrude beyond the proximal end 101 of the fork head 10.

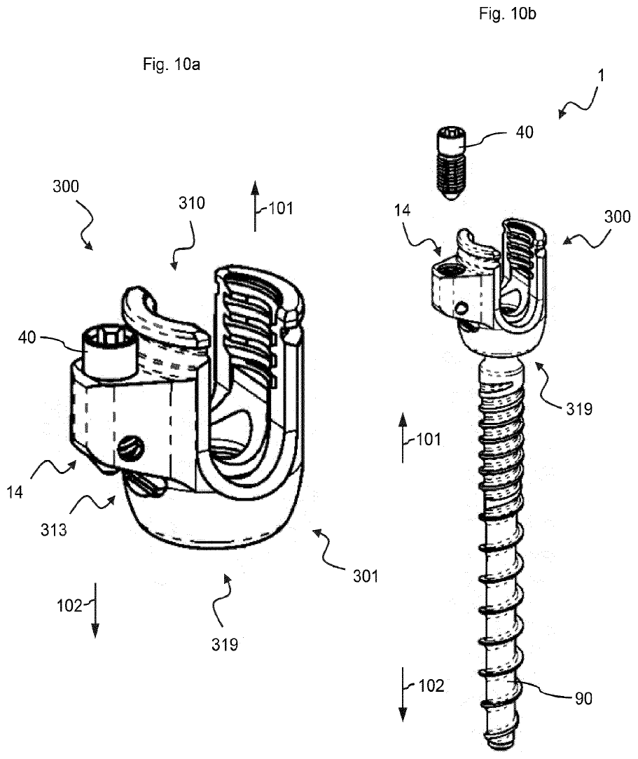

FIG. 10a shows an attachment system 300 in an isometric view. The proximal end of the attachment system 300 is aligned upward according to a proximal direction 101 and its distal end is aligned downward according to a distal direction 102. The attachment system 300 has a main body 301. Proximally, the main body 301 has a first receiving region 310. Distally, the main body 301 has a second receiving region 319. Laterally distally, the main body 301 has a third receiving region 313. Laterally proximally, the main body 301 has an axial opening 14. A positioning element 40 is received in the axial opening 14.

FIG. 10b shows an osteosynthesis device 1 with the attachment system 300 depicted in FIG. 10a in an isometric view. The positioning element 40 is depicted spaced from the axial opening 14 along the proximal direction 101 so that the proximal entrance of the axial opening 14 is visible. A bone anchor 90 is received in the second receiving region 319 in the neutral position. Comparing FIGS. 10a and 10b, it can be seen that the attachment system 300 is provided in a modular manner, i.e. the bone anchor 90 can be fitted in or snapped in modularly (FIG. 10b) and removed again (FIG. 10a).

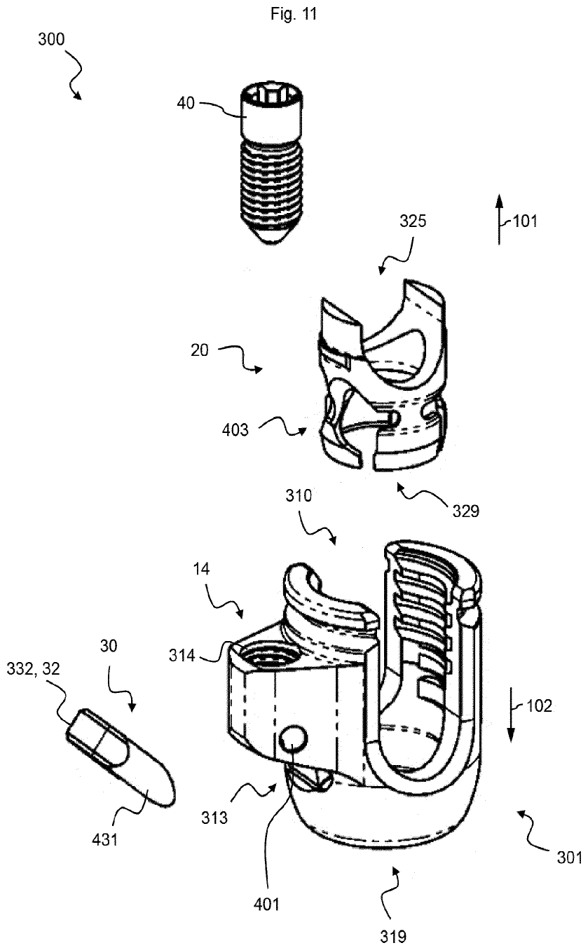

FIG. 11 shows the attachment system 300 depicted in FIGS. 10a and 10b in an exploded view. The attachment system 300 has a thrust piece 20 that is depicted in the distal direction 101 above the first receiving region 310 of the main body 301. In the proximal direction 101, the thrust piece 20 has a first receiving region 325, which is designed to be equivalent to the first receiving region 310 of the main body 301. In the distal direction 102, the thrust piece 20 further has a second receiving region 329, which is designed to be equivalent to the first receiving region 310 of the main body 301. In addition, the thrust piece 20 laterally has a third receiving region 403, which is designed to be equivalent to the third receiving region 313 of the main body 301. The axial opening 14 has an inner thread 314, the proximal region of which can be seen here. The axial opening 14 further has a compression opening 401, which is positioned at the distal end of the inner thread 314—it can only be seen here that the compression opening 401 is positioned at the distal end of the axial opening 14 and that the inner thread 314 is no longer present in the axial opening 14 at the distal-proximal height. The attachment system 300 further has a fixing element 30. The fixing element 30 has a load receiving region 332 or contact region 32 laterally or proximally. The fixing element 30 further has a first fixing pin 431.

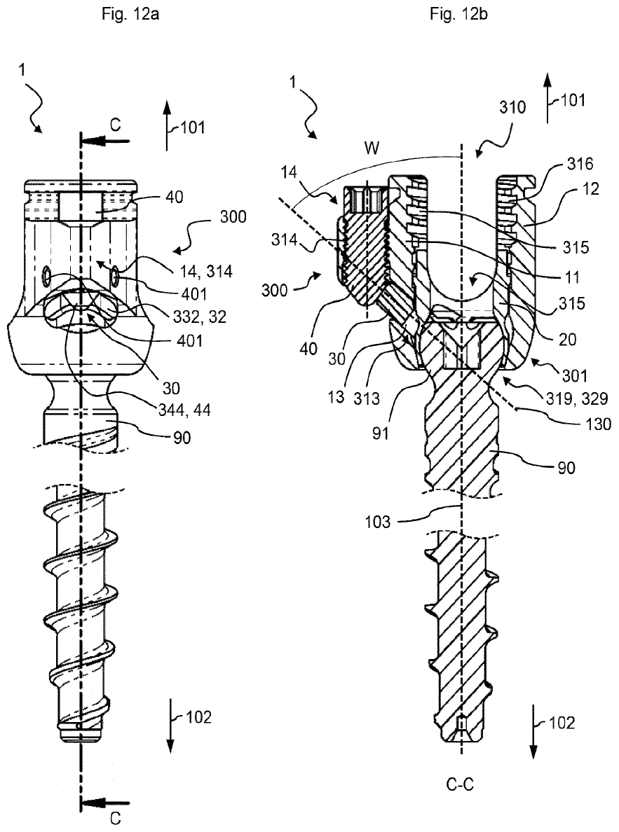

FIG. 12a shows the osteosynthesis device 1 depicted in FIG. 10b in a side view with a sectional plane. The sectional plane runs centrally through the osteosynthesis device 1 and centrally through the positioning element 40 and the bone anchor 90. The axial opening 14 further has a second compression opening 401. Both compression openings 401 are each positioned laterally at the axial opening 14. Through these two compression openings 401, the distal region of the inner thread 314 of the axial opening 14, for example, its last thread turn, can be compressed.

Distally (see distal direction 102), the positioning element 40 has a load introduction region 344 or contact region 44. The load introduction region 344 is in contact or engagement with the load receiving region 332 of the fixing element 30. The axial opening 14 is delimited along the distal direction 102 or the distal end is positioned such that the second receiving region 319 of the main body 301 is accessible from the outside. If the positioning element 40 is screwed into the axial opening 14 to such an extent that the proximal end of the positioning element 40 comes in proximity to the proximal end of the axial opening 14, the distal end of the positioning element 40 protrudes from the axial opening 14, i.e. is then positioned further along the distal direction 102 than the distal end of the axial opening (compare also FIG. 12b). In this way, the positioning element 40 acts via its distal end as a loss prevention device for the fixing element 30.

FIG. 12b shows the osteosynthesis device 1 depicted in FIGS. 10b and 12a in a sectional view C-C along the sectional plane also depicted in FIG. 12a. The sectional plane runs centrally through the osteosynthesis device 1 and centrally through the positioning element 40 and the bone anchor 90. The thrust piece 20 is received in the first receiving region 310 in such a way that the first receiving region 310 of the main body 301 and the first receiving region 325 of the thrust piece 20 as well as the second receiving region 319 of the main body 301 and the first receiving region 329 of the thrust piece 20 complement one another. Along the proximal direction 101, the main body 301 has a first fork leg 11 having a threaded portion 315 and a second fork leg 12 having a threaded portion 316. Both threaded sections 315 and 316 are designed to be complementary to one another. Furthermore, a central axis 103 is depicted, which runs centrally through the main body 301 or its first receiving region 310 and the second receiving region 319 of the main body 301 and centrally through the second receiving region 329 of the thrust piece 20 and the bone anchor 90 positioned in the neutral position. Also depicted is a fixing element axis 130 that runs longitudinally along the third receiving region 313 and the fixing element 30 received therein. The bone anchor 90 has a head region 91 in the proximal direction (compare proximal direction 101). The bone anchor 90 or its head region 91 is received in the second receiving region 329 of the thrust piece and in the second receiving region 319 of the main body 301 in the attachment system 300. The third receiving region 313 has a transverse opening 13 in which the fixing element 30 is received or mounted. The third receiving region 313 is arranged offset with respect to the first receiving region 310 and the second receiving region 319, that is deviating from the axis resulting from proximal to distal. The offset is depicted here on the basis of the rotation of the fixing element axis 130 about a point in the head region 91 or in the second receiving region 319 of the main body 301 in the sectional plane C-C. In this case, the central axis 103 and the fixing element axis 130 form an angle W. If the positioning element 40 strikes the fixing element 30 from the proximal end, the angle W can theoretically be in the range from 0° to 90°, wherein it is preferably in the range from 20° to 80°. The smaller the angle W is, the smaller the space requirement is, in particular for the axial opening 14 and the third receiving region 313 of the main body 301. The larger the angle W is, the greater the clamping effect is that can be generated with the fixing element 30 on the bone anchor 90 or its head region 91.

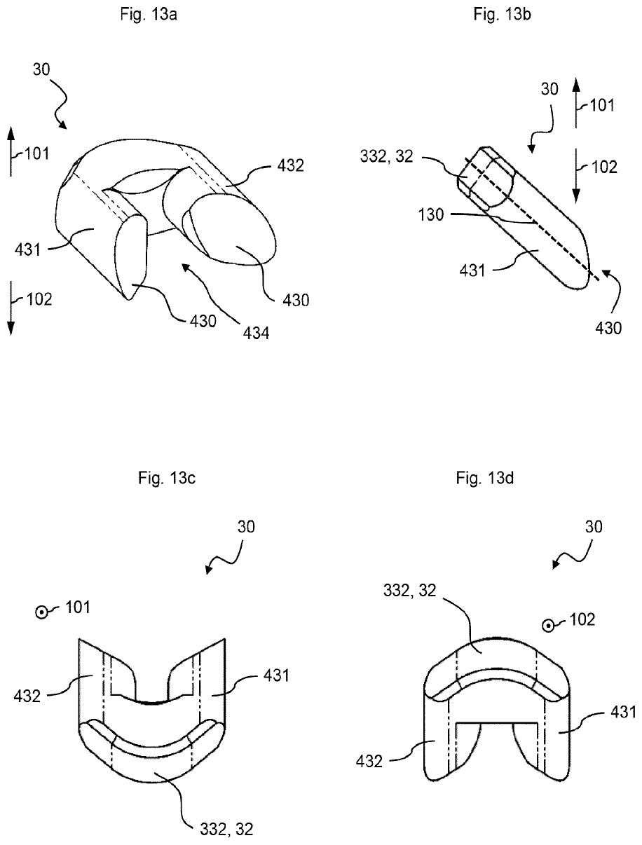

FIG. 13a shows a fixing element 30 with a first fixing pin 431 and a second fixing pin 432 in an isometric view. The first fixing pin 431 and the second fixing pin 432 are each designed rounded toward the outside. The first fixing pin 431 and the second fixing pin 432 each have a contact surface 430 at their distal end. A clearance 434 is located between the first fixing pin 431 and the second fixing pin 432.

FIG. 13b shows the fixing element 30 depicted in FIG. 13a in a side view. The fixing element 30 is aligned here distally-proximally as depicted in FIGS. 12a and 12b. The contact surfaces 430 are configured here chamfered, so that despite the fixing element 30 being offset by the angle W (compare FIG. 12b), these contact surfaces 430 touch the bone anchor 90 or its head region 91 over a large region. Furthermore, the contact surfaces 430 are chamfered inwards, that is in the direction of the clearance 434. Spherically concavely curved contact surfaces 430 are generated by both of the aforementioned chamfers, that is inwards and in the proximal direction 101 or distal direction 102, which contact surfaces 430 irrespective of the rotational position of the bone anchor 90 conform to the head region 91 thereof over a large region. For a different geometry of the head region 91, the contact surfaces 430 can be correspondingly differently curved. The load receiving region 332 or contact region 32 depicted on the left is formed or oriented substantially perpendicular to the main direction of the fixing element 30, that is perpendicular to the fixing element axis 130. The same applies to the load introduction region 344 or contact region 44 of the positioning element 40—this is also formed perpendicularly to the fixing element axis 130 (see FIG. 12b). Accordingly, the load receiving region 332 and the load introduction region 344 are positioned substantially parallel to one another, so that the largest possible load transmission region and thus the safest possible load transmission results. The contact surfaces 430 may be formed with an increased surface roughness, as is the case, for example, with the contact region 31 of the fixing element 30 depicted in FIGS. 5a and 5b.

FIG. 13c shows the fixing element 30 depicted in FIGS. 13a and 13b in a plan view. FIG. 13d shows the fixing element 30 depicted in FIGS. 13a to 13c from below. The fixing element 30 is aligned distally-proximally in both FIGS. 13c and 13d as depicted in FIGS. 12a, 12b, 13a and 13b.

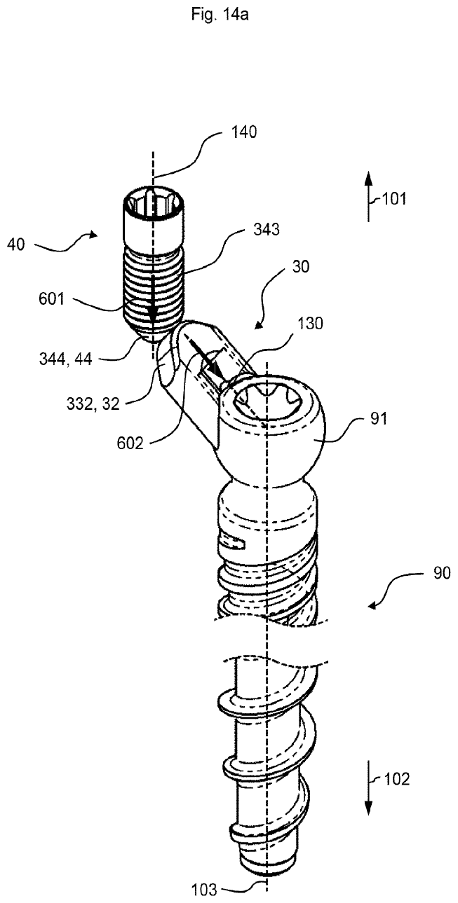

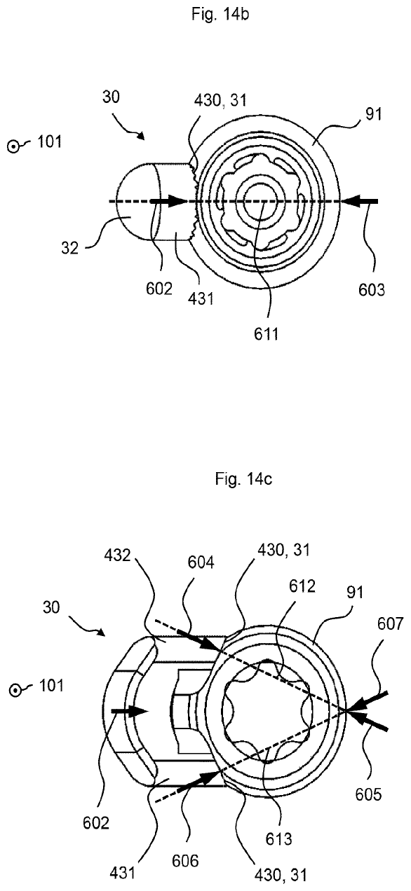

FIG. 14a shows a positioning element 40 in engagement with the fixing element 30 depicted in FIGS. 13a to 13d and a bone anchor 90 in an isometric view. The fixing element 30 has an outer thread 343 that is complementary to the inner thread 314 of the axial opening 14 (compare, for example, FIG. 12b). The positioning element 40 is screwed into the axial opening 14, not depicted here, in such a way that the load introduction region 344 or contact region 44 is in engagement with the load receiving region 332 or contact region 32 (compare, for example, FIG. 12b). In this case, a direction of action 601 results along a positioning element axis 140 with respect to the positioning element 40. This direction of action 601 runs parallel to the central axis 103, since this in turn runs parallel to the axial opening 14 (compare FIG. 12b), and in the same direction as the distal direction 102. Between the load introduction region 344 and the load receiving region 332, the load is transmitted from the positioning element 40 to the fixing element 30, the direction of action 602 resulting from the alignment of the fixing element 30, that is following the fixing element axis 130 with respect to the fixing element 30. According to this direction of action 602, the contact surfaces 430 are pressed onto the bone anchor 90 or its head region 91 (compare FIG. 13a).

FIG. 14b shows a fixing element 30 with a first fixing pin 431 in engagement with a head region 91 of a bone anchor 90 in a plan view. The fixing element 30 is thereby pressed onto the head region 91 in such a way that its first fixing pin 431 or the contact surface 430/the contact region 31 is pressed onto the head region 91 in a contacting manner, resulting in a direction of action 602 that runs essentially perpendicular to the contact surface 430 or the contact region 31. As a result, the head region 91 is pressed into the second receiving region 319 of the main body 301 or into the second receiving region 329 of the thrust piece 20 (both not depicted here, but compare FIGS. 3 and 12b), resulting in a direction of action 603 directed opposed to the direction of action 602. The mutually opposed directions of action 602 and 603 run on an axis of action 611, which runs centrally through the head region 91. In this way, a secure clamping effect is generated via a two-point clamping.

FIG. 14c shows the fixing element 30 depicted in FIGS. 13a to 13d in a plan view in the engagement position depicted in FIG. 14a with the head region 91 of the bone anchor 90. Along the direction of action 602 (which runs as depicted in FIG. 14b), the fixing element 30 is pressed onto the head region 91. At the thereby resulting contact surfaces between the contact surface 430/the contact region 31 of the first fixing pin 431 and the contact surface 430/the contact region 31 of the second fixing pin 432, the direction of action 606 in turn results with respect to the first fixing pin 431 and the direction of action 604 results with respect to the second fixing pin 432. These both directions of action 604, 606 are oriented here substantially perpendicularly to the respective contact surface 430/contact region 31. As a result, the head region 91 is pressed into the second receiving region 319 of the main body 301 or into the second receiving region 329 of the thrust piece 20 (both not depicted here, but compare 12b), resulting in a direction of action 605 (opposed to the direction of action 604), 607 (opposed to the direction of action 606) in each case directed opposed to the directions of action 604, 606. These both directions of action 605, 607 further result on the side of the head region 91 opposed to the starting points of the directions of action 604, 606. The mutually opposed directions of action 604 and 605 as well as 606 and 607 respectively run on an axis of action 612 (corresponding to the directions of action 604 and 605) and 613 (directions of action 606 and 607), which respectively run decentrally through the head region 91. Both directions of action 605 and 607 start in the same region of the head region 91. In this way, a secure clamping effect is generated via a three-point clamping, which is reinforced again compared to the clamping effect depicted in FIG. 14b.

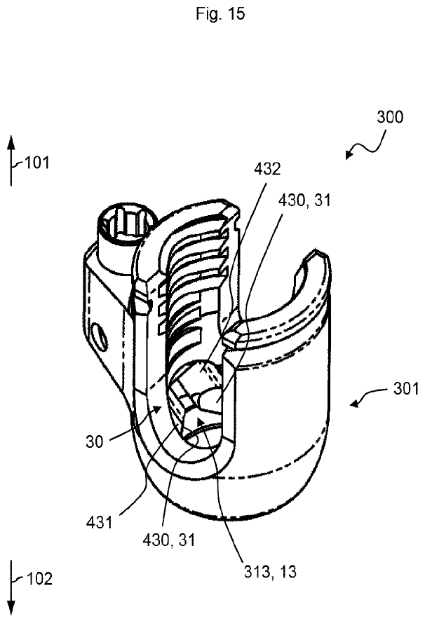

FIG. 15 shows the attachment system 300 depicted in FIG. 10a in another isometric view. No thrust piece 20 is depicted in this representation. Furthermore, the distal end of the third receiving region 313 of the main body 301 or the distal end of the transverse opening 13 is depicted, from which the distal end of the fixing element 30 protrudes or whose contact surfaces 430/contact regions 31 protrude. Following the outer contour of the fixing element 30 (see FIG. 13a), the inner contour of the transverse opening 13 is designed approximately as an elongated hole in cross-section. In order to enable a secure guidance of the fixing element 30, this cross-section of the transverse opening 13 is here constant from proximal to distal (also compare FIG. 12a).

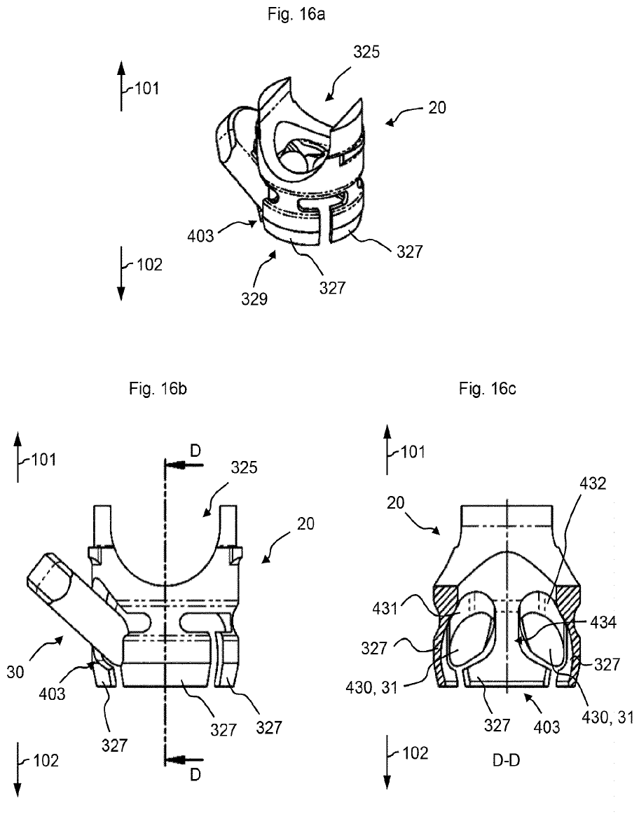

FIG. 16a shows the fixing element 30 depicted in FIGS. 13a to 13d in engagement with a thrust piece 20 in an isometric view. The thrust piece 20 has a plurality of spring elements 327, for example four spring elements 327 distributed radially. The first receiving region 325 of the thrust piece 20 is here designed tapered in the proximal direction 101. In the third receiving region 403 of the thrust piece 20, the fixing element 30 is received in the distal-proximal alignment likewise depicted in FIGS. 12a, 12b, 13a and 13b.

FIG. 16b shows the fixing element 30 depicted in FIG. 16a in the engagement position with the thrust piece 20, likewise depicted in FIG. 16a, in a side view with a sectional plane. Here, too, the fixing element 30 is received in the third receiving region 403 of the thrust piece 20 in the distal-proximal alignment likewise depicted in FIGS. 12a, 12b, 13a, 13b and 16a. Three spring elements 327 can be seen here, wherein the thrust piece 20 further has a fourth spring element 327, which is covered by the spring element 327 depicted in the centre (see FIG. 16c). The spring element 327 depicted on the left is located in the third receiving region 403.

FIG. 16c shows the fixing element 30 depicted in FIGS. 16a and 16b in the engagement position, likewise depicted in FIGS. 16a and 16b, with the thrust piece 20 in a sectional view D-D along the sectional plane likewise depicted in FIG. 16b. In this case, the spring element 327 arranged in the third receiving region 403 is designed in such a way that the first fixing pin 431 and the second fixing pin 432 can slide past it without this spring element 327 entering here into a force closure connection with the first fixing pin 431 and/or the second fixing pin 432. In the distal direction 102, the spring element 327 arranged in the third receiving region 403 thickens below the first fixing pin 431 and the second fixing pin 432. Likewise, the spring element 327 arranged in the third receiving region 403 thickens in the proximal direction 101 above the first fixing pin 431 and the second fixing pin 432. In the view depicted in FIG. 16c, the spring element 327 arranged in the third receiving region 403 covers the clearance 434 or slides into it (compare, for example, FIG. 13a). In this way, the thrust piece 20 is also secured against loss through the fixing element 30.

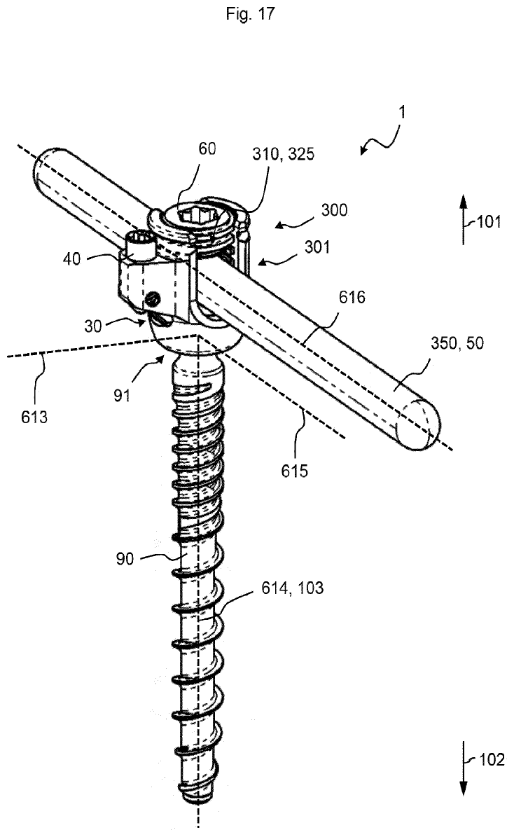

FIG. 17 shows the osteosynthesis device 1 depicted in FIGS. 10b, 12a and 12b with a connecting element 350 in an isometric view. In this case, the connecting element 350 is designed as a connecting rod 50. The connecting rod 50 is rotationally symmetrical. The connecting rod 50 is received in the first receiving region 310 of the main body 301 and in the first receiving region 325 of the thrust piece 20 and is secured or pre-secured by means of a locking element 60. If the locking element 60 were to be fastened further, the connecting rod 50 would be pressed further onto the thrust piece 20 (not visible here, compare FIG. 12b) and ultimately a final fixation of the bone anchor 90 would be achieved. Further, this would result in a final fixation of the connecting rod 50 with respect to the attachment system 300 such that the connecting rod 50 is neither removable from the attachment system 300 in the distal direction 101 nor laterally removable from the attachment system 300 along the longitudinal axis of the connecting rod 50. In particular, lateral fixation with respect to the attachment system 300 is illustrated here by an axis of action 616, which runs parallel to or along the longitudinal axis of the connecting rod 50. However, the state of the pre-fixing is depicted here, as is achieved by fastening the positioning element 40 and thereby pressing the fixing element 30 onto the head region 91 (not visible here, compare FIG. 12b) and results from the combined view of FIGS. 14a and 14c. The three-point clamping depicted in FIG. 14c results in three mutually perpendicular axes of action 613, 614, 615 with respect to the head region 91 or bone anchor 90, which span a clamping effective space within which the bone anchor 90 is or gets pre-fixed with respect to its alignment and position with respect to the attachment system 300. The three axes of action 613, 614, 615 intersect here in the head region 91. The axis of action 614 runs here at least partially congruently to the central axis 103. The axis of action 615 runs parallel to the axis of action 616. The axis of action 613 runs perpendicular to the axes of action 614 and 615 and through the head region 91. The clamping effect achieved by the pre-fixing depicted in FIG. 17 is achieved in particular along the longitudinal axis of the connecting rod 50, which is depicted by the axis of action 615 as described above. In contrast to the two-point clamping depicted in FIG. 14b, the three-point clamping depicted in FIGS. 14c and 17 thereby reinforces in particular the clamping effect along and in the direction of the axis of action 615, i.e. along the longitudinal axis of the connecting rod 50.

Further preferred embodiments are depicted in the following sections 1 to 30.

1. Osteosynthesis device 1, for the treatment of the spine, consisting of or having a fork head 10 which is U-shaped in a side view and has a through opening 18, and the fork head 10 has two fork legs 11, 12 in the proximal direction 101 with an inner-lying thread 16, and a connecting rod 50 can be received therein, and a ball head receiving region 19 is provided in the fork head 10 in the distal direction 102 in the through opening 18, and a bone anchor 90 is pivotably mounted therein, wherein the centre of the inner-lying thread 16 and the centre of the ball head receiving region 19 define a central axis 103 and at least one leg 11, 12 provides an axial opening 14, arranged parallel to the central axis 103, for a positioning element 40, characterized in that a transverse opening 13 is provided in the fork head 10, which communicates with the axial opening 14 and with the through opening of the fork head 18, and a fixing element 30 is movably guided in this transverse opening 13 along an axis 130, and the fixing element 30 clamps the head region 91 of the bone anchor 90 in an angularly stable manner in the fork head 10 with introduction of a compression force along the transverse opening axis 130.

2. Osteosynthesis device 1 according to section 1, characterized in that, when the compression force on the fixing element 30 is released, the head region of the bone anchor 91 in the fork head 10 becomes movable.

3. Osteosynthesis device 1 according to one of the preceding sections, characterized in that a positioning element 40 is guided in the axial opening 14 and, by adjusting the positioning element 40, a compression force is generated, which is transmitted or deflected on the fixing element 30, and thereby a compression force is generated along the transverse opening axis 130, which leads to the temporary clamping of the bone anchor head region 91 in the fork head 10.

4. Osteosynthesis device 1 according to one of the preceding sections, characterized in that a connecting rod 50 can be inserted and by means of a locking element 60 can be fixed to the fork head 10, thereby achieving an angularly stable clamping between the bone anchor head region 91 and the fork head 10, and the angularly stable clamping with the aid of the locking element 60 and the angularly stable clamping with the aid of the fixing element 30 can be activated independently of one another and can also be combined with one another.

5. Osteosynthesis device 1 according to one of the preceding sections, characterized in that the positioning element 40 is part of the osteosynthesis device 1 and remains in the patient after the final locking with the connecting rod 50 and the locking element 60.

6. Osteosynthesis device 1 according to one of the preceding sections, characterized in that the positioning element 40 does not protrude beyond the proximal end of the fork head 10 in the proximal direction 101.

7. Osteosynthesis device 1 according to one of the preceding sections, characterized in that the positioning element 40 is part of the osteosynthesis device 1 and has a predetermined breaking point 45, and only a part of the positioning element 40 remains in the patient after the final locking with the connecting rod 50 and the locking element 60.

8. Osteosynthesis device 1 according to one of the preceding sections, characterized in that the positioning element 40 is part of an instrument and is removed from the patient after the final locking with the connecting rod 50 and the locking element 60.

9. Osteosynthesis device 1 according to one of the preceding sections, characterized in that the fixing element 30 has a radially inwardly directed contact region 31, which abuts directly against the head region 91 of the bone anchor 90, and this contact region 31 is concavely shaped at least in sections in a side view.

10. Osteosynthesis device 1 according to one of the preceding sections, characterized in that the radially inwardly directed contact region 31 of the fixing element 30 approximates, in a side view, at least a section of the outer surface of the bone anchor head region 91.

11. Osteosynthesis device 1 according to one of the preceding sections, characterized in that the radially inwardly directed contact region 31 of the fixing element 30 has an increased roughness, notches or teeth.

12. Osteosynthesis device 1 according to one of the preceding sections, characterized in that the fixing element 30 can assume a position S1 along the transverse opening axis 130, in which a compression force is transmitted on the bone anchor 90, so that the bone anchor 90 is held angularly stable in the ball seat 19, and the fixing element 30 can assume a second position S2, in which the bone anchor 90 is held movably in the ball seat 19.

13. Osteosynthesis device 1 according to one of the preceding sections, characterized in that the fixing element 30 has a radially outwardly directed contact region 32, which is in direct contact with a distal contact region 44 of the positioning element 40, and this contact 32, 44 is configured in such a way that a compression force along the positioning element axis 140 is diverted into a compression force along the fixing element axis 130.

14. Osteosynthesis device 1 according to one of the preceding sections, characterized in that the radially outwardly directed contact region 32 of the fixing element 30 is provided convexly in sections in at least one side view.

15. Osteosynthesis device 1 according to one of the preceding sections, characterized in that the radially outwardly directed contact region 32 of the fixing element 30 has a bevel in at least one side view.

16. Osteosynthesis device 1 according to one of the preceding sections, characterized in that the distal contact region 44 of the positioning element 40 is provided convexly at least in sections.

17. Osteosynthesis device 1 according to one of the preceding sections, characterized in that the axis of the fixing element 130 is directed approximately at the virtual centre of the ball head receiving region 19 and intersects with the central axis 103.

18. Osteosynthesis device 1 according to one of the preceding sections, characterized in that the axis of the fixing element 130 is arranged at an angle W between 10° and 90°, preferably between 30° and 85°, preferably between 60° and 80°, with respect to the central axis 103.

19. Osteosynthesis device 1 according to one of the preceding sections, characterized in that the opening of the fixing element 13 along the fixing element axis 130 has, at least at one point, an indent, undercut, a pin, step, deformable region or taper 131, which is suitable for holding the fixing element 30 captively in this opening 13.

20. Osteosynthesis device 1 according to one of the preceding sections, characterized in that, for the assembly of the osteosynthesis device 1, the fixing element 13, coming from the central through opening 18, is fitted into the transverse opening 13 and, subsequently, the bone anchor 90 is placed with its head region 91 into the through opening 18 in the ball seat 19, and the fixing element 30 is thus held captively in the transverse opening 13.

21. Osteosynthesis device 1 according to one of the preceding sections, characterized in that the axial opening 14 for the positioning element 40 has an inner thread at least in sections.

22. Osteosynthesis device 1 according to one of the preceding sections, characterized in that the transverse opening 13 for the fixing element 30 has an inner thread at least in sections.

23. Osteosynthesis device 1 according to one of the preceding sections, characterized in that the positioning element 40 is designed in the form of a pin and has a tool attachment point 42 in the proximal direction 101.

24. Osteosynthesis device 1 according to one of the preceding sections, characterized in that the positioning element 40 is part of an instrument and the fork head 10 has projections, openings, grooves, bars, profiles or other features 17 which are suitable for gripping, engaging or engaging from behind with an instrument, which serves to introduce a tensile force on the fork head, which acts as an antagonist to the introduction a compression force via the positioning element 40.

25. Osteosynthesis device 1 according to one of the preceding sections, characterized in that the fork head 10 has a thrust piece 20 and the thrust piece has a through opening 28, a distally directed contact region towards the bone anchor head region 29, a proximally directed stave bearing 25 and, in the region of the bone anchor head region 29, a lateral opening or partial cutout 23, in which the fixing element 30 is arranged in a freely movable manner.

26. Osteosynthesis device 1 according to one of the preceding sections, characterized in that the fork head 10 provides a thrust piece 20 and, when the bone anchor head region 91 is clamped solely by the fixing element 30, the thrust piece 20 is unloaded.

27. Osteosynthesis device 1 according to one of the preceding sections 25 or 26, characterized in that the thrust piece 20 has open slots 26 in the distal direction 102 and, as a result, at least three resilient arms 27 are formed on the head receiving region 29 and the resilient arms 27 describe a cone 271 on their outer side at least in sections, and the bone anchor 90 can be fitted into the fork head 10 coming from the distal direction 102.

28. Osteosynthesis device 1 according to section claim 27, characterized in that the fork head 10 defines at least one inner cone section 183 in the ball receiving region 19, which leads congruently to the cone 271 of the thrust piece 20 and, when the locking element 60 is actuated, leads to the angularly stable clamping of the bone anchor head region 91 with the fork head 10.

29. Osteosynthesis device 1 according to one of the preceding sections 27 or 28, characterized in that the fork head 10 in the through opening 18 has a region which, at least in sections, has a larger inner diameter 182 than the core diameter of the thread 16.

30. Osteosynthesis device 1 according to one of the preceding sections, characterized in that the legs 11, 12 each provide a supporting surface 181 with an undercut, which is effective in the proximal direction 101, and the thrust piece 20 has radially outwardly directed projections 24 at the proximal end, the projections 24 being designed to be resilient in the radially inward direction, so that the thrust piece 20, coming from the proximal direction 101, can be fitted into the fork head 10 and the projections of the thrust piece 24 latch with the supporting surfaces 181, and the thrust piece 20 is secured in the proximal direction 101 but not subjected to force.

### LIST OF REFERENCE SIGNS

1 Osteosynthesis device
10 Fork head
11 (First) fork leg
12 (Second) fork leg
13 Transverse opening
14 Axial opening
16 Thread
17 Projections, openings, grooves, bars, profiles or other features
18 Through hole
19 Ball head receiving region
20 Thrust piece
21 Leg (of the thrust piece)
22 Leg (of the thrust piece)
23 Cutout
24 Projection
25 Stave bearing
26 Slot
27 Arm
28 Through hole
29 Bone anchor head region
30 Fixing element
31 Contact region
32 Contact region
40 Positioning element
42 Tool attachment point
43 Thread (of the positioning element)
44 Contact region
45 Predetermined breaking point
50 Connecting rod
60 Locking element
90 Bone anchor
91 Head region (of the bone anchor)
92 Tool attachment point (of the bone anchor)
93 Bone thread
101 Proximal direction
102 Distal direction
103 Central axis
130 Fixing element axis
140 Positioning element axis
181 Supporting surface
182 Inner diameter
183 Inner cone section
271 Cone
300 Attachment system
301 Main body
310 First receiving region (of the main body)
313 Third receiving region (of the main body)
314 Inner thread (of the axial opening)
315 Threaded section (of the first fork leg)
316 Threaded section (of the second fork leg)
319 Second receiving region (of the main body)
323 Transverse opening (of the thrust piece)
325 First receiving region (of the thrust piece)
327 Spring element
329 Second receiving region (of the thrust piece)
332 Load receiving region
343 Outer thread (of the positioning element)
344 Load introduction region
350 Connecting element
352 Longitudinal axis (of the connecting element)
401 Compression opening
403 Third receiving region (of the thrust piece)
430 Contact surface (of the fixing pin)
431 First fixing pin
432 Second fixing pin
434 Clearance
501 First opening (of the transverse opening)
502 Second opening (of the transverse opening)
601 Direction of action
602 Direction of action
603 Direction of action
604 Direction of action
605 Direction of action
606 Direction of action
607 Direction of action
611 Axis of action
612 Axis of action
613 Axis of action
614 Axis of action
615 Axis of action
616 Axis of action
S1 Position
S2 Position
W Angle

1. An attachment system for an osteosynthesis device for spinal treatment, having a main body, comprising:
a first receiving region which is designed for receiving a connecting element,
a second receiving region which is designed for receiving a bone anchor,
a third receiving region having a linear transverse opening, the transverse opening having a first opening a its first end and a second opening at its second end, and
an axial opening for receiving and guiding a positioning element,
the device further having a fixing element having a first fixing pin, and a load receiving region, wherein the first fixing pin is insertable into the transverse opening via the first opening and the fixing element is longer than the transverse opening, and wherein the first fixing pin has a contact surface at its end remote from the load receiving region, and
a positioning element having a load introduction region, wherein the positioning element can be positioned and guided by means of the axial opening in such a way that the fixing element can be guided along the transverse opening in the direction of the second receiving region by means of the pressure exerted by the load introduction region on the load receiving region.

2. The attachment system according to claim 1, wherein the third receiving region is configured in such a way that the transverse opening is provided with an offset with respect to the central axis of the bone anchor received in the second receiving region in the neutral position, the offset being at least 20° and a maximum of 80°.

3. The attachment system according to claim 1, wherein the load introduction region and the load receiving region are designed to be complementary to one another.

4. The attachment system according to claim 1, wherein the attachment system further comprises a thrust piece, wherein the thrust piece has a first receiving region equivalent to the first receiving region of the attachment system, a second receiving region equivalent to the second receiving region of the attachment system and having a plurality of spring elements, and a third receiving region equivalent to the third receiving region of the main body and having a linear transverse opening, and wherein the thrust piece can be inserted into the main body via the first receiving region of the main body.

5. The attachment system according to claim 1, wherein the axial opening has an inner thread and the positioning element has an outer thread, and wherein the inner thread of the axial opening is designed to be complementary to the outer thread of the positioning element, in particular wherein the inner thread of the axial opening runs substantially parallel to the central axis of the bone anchor received in the second receiving region in the neutral position.

6. The attachment system according to claim 5, wherein the axial opening has at least one compression opening, wherein the compression opening is positioned at the distal end of the inner thread, and wherein the compression opening is designed in such a way that it allows the outer thread of the positioning element to be compressed.

7. The attachment system according to claim 1, wherein the fixing element further comprises a second fixing pin.

8. The attachment system according to claim 1, wherein the contact surface of the first fixing pin or optionally of the first and/or second fixing pin is designed to be complementary to a corresponding contact surface of the bone anchor.

9. The attachment system according to claim 7, wherein a spring element is arranged in the linear transverse opening of the thrust piece, and wherein this spring element is placed in particular centrally in the linear transverse opening.

10. The attachment system according to claim 9, wherein the spring element arranged in the linear transverse opening is designed for widening in the distal region.

11. The attachment system according to claim 1, wherein the contact surface of the first fixing pin or optionally of the first and/or second fixing pin has a locally increased surface roughness.

12. The attachment system according to claim 1, wherein the first receiving region is designed as a fork head, in particular as a U-shaped fork head, which has a first fork leg and a second fork leg.

13. The attachment system according to claim 1, wherein the first receiving region of the main body has a thread.

14. The attachment system according to claim 12, wherein the third receiving portion and the axial opening are disposed on the first fork leg.

15. An osteosynthesis device for spinal treatment, having a bone anchor and the attachment system according to claim 1.

16. An osteosynthesis device, for the treatment of the spine, consisting of or having a fork head which is U-shaped in a side view and has a through opening, and the fork head has two fork legs in the proximal direction with an inner-lying thread, and a connecting rod can be received therein, and a ball head receiving region is provided in the fork head in the distal direction in the through opening, and a bone anchor is pivotably mounted therein, wherein the centre of the inner-lying thread and the centre of the ball head receiving region define a central axis and at least one leg provides an axial opening, arranged parallel to the central axis, for a positioning element, wherein a transverse opening is provided in the fork head, which communicates with the axial opening and with the through opening of the fork head, and a fixing element is movably guided in this transverse opening along an axis, and the fixing element clamps the head region of the bone anchor in an angularly stable manner in the fork head with introduction of a compression force along the transverse opening axis.

17. The osteosynthesis device according to claim 16, wherein the transverse opening in the fork head is provided transversely to the axial opening and the central axis, and wherein the compression force introduced along the transverse opening axis is generated by the positioning element.

18. The osteosynthesis device according to claim 16, wherein the radially inwardly directed contact region of the fixing element has an increased roughness, notches or teeth.

19. The osteosynthesis device according to claim 16, wherein the transverse opening for the fixing element has an inner thread at least in sections.

20. The osteosynthesis device according to claim 16, wherein the fork head has a thrust piece OK and the thrust piece has a through opening, a distally directed contact region towards the bone anchor head region, a proximally directed stave bearing, and, in the region of the bone anchor head region, a lateral opening or partial cutout, in which the fixing element is arranged such that it can move freely.

21. The osteosynthesis device according to claim 16, wherein the fork head provides a thrust piece and, when the bone anchor head region is clamped solely by the fixing element, the thrust piece is unloaded.
